(19) United States
(12) Patent Application Publication
(10) Pub. No.: US 2008/0064917 A1
(43) Pub. Date: Mar. 13, 2008

(54) Amplification-Based Cardiac Assist Device

(76) Inventors: Eli Bar, Moshav Megadim (IL); Benny Rousso, Rishon LeZion (IL); Ran Kornowski, Ramat Hasharon (IL)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 10/575,312

(22) PCT Filed: Oct. 15, 2004
(86) PCT No.: PCT/IL04/00950

§ 371 Date: Feb. 22, 2007

Related U.S. Application Data
(60) Provisional application No. 60/511,548, filed on Oct. 15, 2003.
(60) Provisional application No. 60/599,176, filed on Aug. 4, 2004.

(51) Int. Cl. A61M 1/12

(52) U.S. Cl. 600/16

(57) ABSTRACT

Apparatus (20) is provided for compressing at least one ventricle of a heart (40). The apparatus includes a plurality of inflatable elements (64) and a pump (24) in fluid communication with the inflatable elements. At least one band (68) is in mechanical communication with the inflatable elements. A portion of the band is adapted to be placed around at least a portion of the heart in mechanical communication with the portion of the heart. The inflatable elements are arranged such that when the inflatable elements are inflated by the pump, the inflatable elements apply more force to the heart via shortening of the portion of the band than via expansion of the inflatable elements against the heart.

### CROSS-REFERENCES TO RELATED APPLICATIONS

The present patent application claims priority from:

(a) U.S. Provisional Patent Application 60/511,548 to Kornowski and Bar, filed Oct. 15, 2003, entitled, “Dynamic external myocardial stent to enhance left ventricular contractility in heart rate patients,” and

(b) U.S. Provisional Patent Application 60/599,176 to Rousso and Bar, filed Aug. 4, 2004, entitled, “Balloon based cardiac assist device.”

Both of these applications are incorporated herein by reference.

### BACKGROUND OF THE INVENTION

Congestive Heart Failure (CHF) occurs when the heart is unable to meet the hemodynamic needs of the body. The contractility of the heart is reduced, and thus the stoke volume and cardiac output are reduced. Cardiac assist devices assist the heart to pump blood in order to meet the body's demand.

The following references may be relevant to the practice of some embodiments of the present invention:

U.S. Pat. No. 6,626,821 to Kung et al., which is incorporated herein by reference, describes a flow-balanced cardiac wrap that assists the right and left ventricles of an affected heart to differing and adjustable degrees. The wrap generally applies an assist to the left ventricle that is greater than that applied to the right, or that reduces blood output from the right relative to the left. In one embodiment, the wrap comprises a material covering that is applied around the right and left ventricles of the heart, so that the left ventricle is assisted over a larger surface area than the right. The positioning of the right ventricular portion or the wrap is chosen to achieve desired pumping characteristics for the right ventricle.

U.S. Pat. No. 6,123,724 to Denker, which is incorporated herein by reference, describes a device that employs electromagnetic force for artificially contracting a heart of a subject to pump blood. The device includes electromagnetic coils attached to ribs of the subject, and permanent magnets placed adjacent the electromagnetic coils. When direct electric current is applied to the electromagnetic coils, the magnetic fields from the coils and the permanent magnets interact to repel the permanent magnets which apply contraction force to the heart.

U.S. Pat. No. 6,099,460 to Denker, which is incorporated herein by reference, describes a method for artificially contracting a heart to pump blood by applying separate electromagnets to the exterior surface of the heart and implanting another electromagnet inside a heart chamber. Electric currents are selectively applied to the electromagnets thereby producing magnetic fields which attract the electromagnets. The attraction generates forces which contract chambers of the heart and pump blood from those chambers. A technique for generating an electric current from a natural contraction of the heart is also described.

U.S. Pat. No. 5,098,369 to Heilman et al., which is incorporated herein by reference, describes a ventricular assist device that includes a cardiac compression assembly which comprises a gel-filled pad of generally concave configuration, mounted on a pressure plate with peripheral portions of the pad extending beyond the periphery of the plate, to preclude damage to the heart by the peripheral edges of the plate. The gel-filled pad may have undulating opposite sides formed by intersecting rows of raised dimples. The pad also includes portions for suturing the pad to a heart ventricle, and at least some of the dimples on the side of the pad facing the heart ventricle are provided with ventricle tissue growth-promoting islands. An electrode, in the form of a grid having intersecting strips which define dimple-receiving openings therebetween, also may be mounted on the ventricle side of the pad. As many as eight circumferentially arranged cardiac compression assemblies, having lower ends pivotally mounted on a support member adapted to be located adjacent the apex of a heart ventricle, may be provided. Operating systems for operating the cardiac compression assemblies may include a motor-driven camming mechanism; a mechanism comprising a device for converting electrical energy to hydraulic fluid energy, two sealed fluid systems, a reversible pump, two bellows and a safety solenoid pump-bypass fluid return valve; or a closed loop system comprising a reversible pump in a fluid supply casing, a hydraulic fluid manifold including a plurality of miniature fluid actuators which may be of arcuate construction to conserve space, and a mechanism for collecting fluid leaking from the actuators and returning it to the fluid supply casing.

U.S. Pat. No. 5,383,840 to Heilman et al., which is incorporated herein by reference, describes a ventricular assist device for a heart that includes a compression band-stay-pad assembly for encircling substantially the entire heart perimeter and comprising an elongated band member or chain disposed in a sealed protective structure filled with a lubricating medium. The band member may be fixed at one end and wound upon, or unwound from, a rotatable spool by a drive motor through a speed reducer. Force-transmitting support or stay assemblies are disposed in the protective structure between the band member and a resilient pad assembly for encircling the heart and promoting heart tissue ingrowth therein. The force-transmitting stay assemblies are biased circumferentially, and thus radially outward, by compression return springs disposed therebetween. The resilient pad assembly includes a corrugated surface provided with vertical coil springs, which help prevent damage to heart tissue and facilitate return of the pad assembly to an initial condition, embedded defibrillator electrodes and relatively soft portions to prevent damage to coronary arteries. A net structure suspended below the device supports the apical portion of the heart.

U.S. Pat. No. 3,464,322 to Pequignot, which is incorporated herein by reference, describes a deformable diaphragm for producing impulsing or pumping effects in a fluid. The diaphragm is formed by a tube of elliptical section wound in a spiral with adjacent turns welded together and the outer edge being gripped in a support. The tube is connected to a source of fluid under pressure, the admission of which causes deformation of the tube and consequent inflation of the diaphragm.

U.S. Pat. No. 5,456,715 to Liotta, which is incorporated herein by reference, describes an implantable mechanical system for assisting blood circulation using a blood circulation pump. The system is actuated by the power produced by the linear contraction of skeletal muscle. The system comprises for its two-phase application: a combined prosthesis defining the bio-mechanical coupling between the skeletal muscle and the implantable mechanical system, and a muscle action force multiplier transmitting force through a lever system driving compression plates of the blood chamber formed into the pump. The biomechanical coupling, the force multiplier, and the lever system form a functional unit interconnected by means of lead wires for transmitting movement. The system further comprising a device for measuring force and the displacement of the skeletal muscle driving the pump, during the electro-stimulation period through the system.

U.S. Pat. No. 4,304,225 to Freeman, which is incorporated herein by reference, describes an auxiliary pumping device for attachment to a portion of a body organ, e.g., a heart, for compressing and releasing the organ alternatingly in response to a series of timing pulses. The pumping device includes a compressor which has an opening therein for receiving and at least partially surrounding the body organ. The compressor is movable periodically to reduce substantially and forcibly the cross-sectional area of the opening by a predetermined amount to squeeze the surrounded portion of the body organ to force body materials therefrom. The pumping device includes an electrical force producing device, such as an electrical motor or a pump, which responds to the timing pulses for applying force to the compressor to cause it to reduce substantially the cross-sectional area of the opening against the force of the body organ being squeezed upon the occurrence of each one of the pulses and for releasing the compressor to permit the body organ to expand rapidly back to its unstressed normal size and shape during the time intervals between the pulses.

U.S. Pat. No. 6,616,596 to Milbocker, which is incorporated herein by reference, describes a unified, non-blood contacting, implantable heart assist system, which surrounds the natural heart and provides circumferential contraction in synchrony with the heart's natural contractions. The pumping unit is composed of adjacent tube pairs arranged along a bias with respect to the axis of the heart and bound in a non-distensible sheath forming a heart wrap. The tube pairs are tapered at both ends such that when they are juxtaposed and deflated they approximately follow the surface of the diastolic myocardium. Inflation of the tube pairs causes the wrap to follow the motion of the myocardial surface during systole. A muscle-driven or electromagnetically powered energy converter inflates the tubes using hydraulic fluid pressure. An implanted electronic controller detects electrical activity in the natural heart, synchronizes pumping activity with this signal, and measures and diagnoses system as well as physiological operating parameters for automated operation.

U.S. Pat. No. 5,713,954 to Rosenberg et al., which is incorporated herein by reference, describes an artificial implantable heart assist system with an artificial myocardium, which employs a number of flexible, non-distensible tubes with the walls along their long axes connected in series to form a cuff. The tubes are sealed for purposes of inflation and deflation with either hydraulic fluid or pneumatic fluid. The cuff is placed around the natural heart. The inflation of the tubular segments provides that they have a circular cross-section, while in the deflated, or collapsed position without being fluid filled, they are essentially flat sheets. The difference in the perimeter length of the cuff in the plane of the tube short axis, arising from the fact that, inflated, each tube has a length along its perimeter equal to the diameter of the inflated tube, while deflated it has a length equal to the perimeter of the tube divided by two, provides for a contractile force. An energy converter is provided in the system for shuttling fluid between a compliant reservoir and the cuff in phase with the systolic and diastolic phase of the natural heart. This system is powered by an internal implanted battery, which can be recharged transcutaneously from an external power source.

U.S. Pat. No. 3,587,567 to Schiff, which is incorporated herein by reference, describes a mechanical ventricular assistance apparatus that comprises a ventricular assistor cup having a configuration generally conforming to the surface configuration of the ventricles of the heart. The cup is formed of a rigid or nonresilient material and includes a flexible liner or diaphragm. The rigid outer shell is provided with an open end for receiving the ventricles and first and second ports for selective coupling to pressure or vacuum systems. The diaphragm is secured about the open end and is further secured adjacent one of the two ports. At least one electrode is provided in the region of one of such ports for the application of signals to carry out fibrillation or defibrillation of the heart. In the case where it is desired to provide mechanical assistance of the heart pumping action in synchronism with normal heart rhythm the electrode may be employed for monitoring the electrocardiac signals and hence for operating the mechanical pumping action in synchronism with the normal heart rhythm.

U.S. Pat. No. 6,406,422 to Landesberg, which is incorporated herein by reference, describes a system for ventricular-assist of the normal heart action, which utilizes an intraventricular device with a limited volume which is expanded at a critical time, for a critical duration and with a volume change course such that it assists the pumping action of the heart without inducing stretching of the ventricular wall.

U.S. Pat. No. 6,238,334 to Easterbrook, III et al., which is incorporated herein by reference, describes a ventricular cuff for assisting a heart to pump blood by applying uniform pressure to a majority portion of an exterior ventricular surface of the heart. A heart engaging structure is preferably provided for releasably engaging the heart to hold the heart in place relative to the cuff. The ventricular cuff includes an outer shell, an inflatable inner bladder and a fastener assembly. The heart engaging structure and ventricular cuff define an upwardly opening chamber sized for receiving a heart. The bladder has an opening for communication with a source of fluid under pressure so that the bladder is cyclically inflated and deflated at a predetermined rate to assist the ventricles of the heart to properly contract.

European Patents EP 0583012B1 and EP 0280301B1 to Heilman, which are incorporated herein by reference, describe a device for compressing a ventricle of a heart from one or more sides in synchronism with the natural contraction of the ventricle (systole), and providing arrhythmia control of the heart. The device is completely implantable in the body of a patient user externally of the heart. Compression of the ventricle is produced by a plurality of spaced compression plate assemblies and a ventricle apex-compression plate, a single compression plate-band assembly or tightenable bands. The compression plate assemblies comprise electrodes for heart monitoring purposes.

US Patent Application Publication 2001/0003802 to Vitale, which is incorporated herein by reference, describes a magnetic spring including a plurality of spaced-apart stationary circumferentially magnetized segments disposed along a circle about an axis to define a first plurality of spaced-apart gaps, and a plurality of spaced-apart moveable circumferentially magnetized segments disposed along the circle to define a second plurality of spaced-apart gaps. Each of the plurality of moveable magnetized segments is axially slidable within a respective one of the first plurality of gaps defined by the plurality of stationary magnetized segments. Applications of the magnetic spring include an actuator of a ventricle assist device (VAD) or a total artificial heart (TAH) in which stored energy in the magnetic spring is used to reduce motor power loses of an actuator during a power stroke of the VAD or TAH.

US Patent Application Publication 2001/0041821 to Wilk, which is incorporated herein by reference, describes a surgical method for assisting cardiac function utilizing a balloon initially in a collapsed configuration. The balloon is inserted into an intrapericardial space about a patient's heart and is disposed about one portion of the patient's heart. The method further includes inflating the balloon in the intrapericardial space to compress one portion of the patient's heart. A lower end portion of the patient's heart is separately compressed by an additional instrumentality to reduce ventricular volume.

PCT Publication WO 02/28450 to Ortiz, which is incorporated herein by reference, describes heart support and assist devices for supporting and assisting the pumping action of the heart. Various embodiments include mesh support devices, devices using straps, spiral-shaped devices, and catheter-based devices.

### SUMMARY OF THE INVENTION

In some embodiments of the invention, one or more shape-changing members are placed adjacent to the heart of a patient. The shape-changing members are coupled to a band that surrounds the heart. Typically, each shape-changing member is surrounded by a respective portion of the band. For example, the band may be looped around the shape-changing member. In this manner, expansion of any given shape-changing member causes a greater length of the band to transiently surround the shape-changing member. (By way of illustration and not limitation, the greater length may be about 15-20 mm.) Since the band itself is typically of substantially fixed length, the remainder of the band that is not surrounding the given shape-changing member is shortened. This shortening of the remaining portion of the band applies a compressive force to the heart, supporting the function of the heart by ejecting blood therefrom during systole. When each of the shape-changing members is expanded generally simultaneously, the total expansion of the shape-changing members is about 1 to about 15 cc, and the band is shortened typically by about 1 cm to about 10 cm, for example, by about 2 cm to about 6 cm. Shortening of the band by this amount typically causes the local circumference of the heart to shorten by essentially the same amount, whereby about 40 cc to about 80 cc are expelled from the heart.

Typically, but not necessarily, a total of about 3 to about 25 shape-changing members surround the heart. In another example, about 5 to about 15 shape-changing members surround the heart. Typically, but not necessarily, the band and all of the shape-changing members are placed within a sleeve, and the sleeve is placed around the heart during a surgical procedure.

In some embodiments of the invention, the shape-changing members comprise balloons. Alternatively or additionally, the shape-changing members comprise piston and cylinder arrangements.

In some embodiments of the present invention, the sleeve comprises an inert material that prevents tissue growth from reaching the shape-changing members. Such a material may be flexible. In some embodiments of the present invention, the sleeve comprises a material that inhibits tissue growth (for example, a steroid-eluting material), particularly within the body of the sleeve. For some applications, the heart-contacting surface of the sleeve is configured in order to enhance its contact with the surface of the heart, for example by means of a chemical tissue-growth facilitator, roughening the heart-contact surface, and/or a mechanical coupler (such as a suture or a hook that securely engages the myocardium).

In some embodiments of the present invention the sleeve surrounds one or more of the heart's chambers, typically both ventricles. For some applications, the sleeve surrounds three chambers (e.g., two ventricles and one atrium) or all four chambers of the heart. In an embodiment, the sleeve surrounds two or more of the heart's chambers, but applies compressive force to the chambers asymmetrically. For example, the sleeve may apply more compressive force to the left ventricle than to the right ventricle. For some applications, the asymmetric force application is attained by configuring the sleeve to have an asymmetric distribution of the shape-changing members.

The sleeve is typically adapted at the time of implantation in order to fit the heart's perimeter. Such adaptation is attained, for example, by changing the length of some of the band portions between adjacent shape-changing members, or by modulating the initial volume or size of one or more of the shape-changing members.

In some embodiments of the present invention, the apex of the heart may be at least partially covered by a substantially stiff structure suitable for minimizing bulging of the apex during ventricular compression. For other applications, bulging of the apex is generally not a concern (e.g., because intracardiac pressure is not excessive), and the apex is covered by a more flexible material (e.g., a mesh), or is not covered at all.

For some applications, the sleeve is configured to support filling of one or more heart chambers during diastole. For example, fluid that is actively driven into the shape-changing members in order to facilitate contraction of the heart may be actively withdrawn during diastole and used to fill one or more diastole-supporting compartments, whereby the filling of the diastole-supporting compartments causes an increase of blood flow into the heart. In an embodiment, the diastole-supporting compartments may change shape due to the active filling thereof. Alternatively or additionally, the sleeve comprises one or more elastic elements, which store energy in association with the increase in size of the shape-changing members during systole. The elastic elements release this energy during diastole, which energy release is directed to cause an outwardly-directed force to be applied to the epicardium, thereby increasing blood flow into the heart.

In accordance with an embodiment of the present invention, an experimental prototype of a cardiac assist device was built, comprising a hydraulic system comprising (a) a pump and (b) a sleeve containing eight balloons. Full details of this experiment may be found in the above-cited US provisional application entitled, “Balloon based cardiac assist device,” which is incorporated herein by reference.

The hydraulic system was placed around a latex heart model. The system was tested at physiological heart rates, pressures and flow rates. Power consumption was measured. DC power of approximately 10 Watts generated reasonable “cardiac” output from the passive latex heart, during a short test period. This power consumption was achieved with the device wet, in order to reduce friction.

The experimental prototype is designed to move approximately 10 cc of water in every beat, in order to pump approximately 20-30 cc of water from the latex heart, at a peak pressure of greater than 110 mmHg. Diastolic pressure was generally maintained between 75 and 80 mmHg. The latex heart has an external diameter of 12 cm, and a base-to-apex length of 10 cm (typical values for end-stage heart failure).

Each balloon is 40 mm in length, and is enclosed within its own flexible latex cover having a similar shape. The sleeve containing the eight balloons has a diameter of about 120 mm during the diastolic phase, and reduces its diameter by up to about 20 mm during the systolic phase. In order to achieve this change, while the heart varies from a diastolic pressure of about 80 mmHg to a systolic pressure of about 120 mmHg, the eight balloons inflated from an initial diameter of 5 or 6 mm to a final diameter in the range of 10 to 12 mm.

The volume of fluids required to cause the change in volume of the balloons is approximately 10 cc, and the inflation pressure was typically in the range of 3-5 atmospheres.

The balloons are connected to a band, which comprises fourteen parallel 0.9 mm diameter Nylon wires. The wires are maintained in alignment by passing through holes in alignment bars disposed next to each balloon. The wires interdigitate as they wrap around each balloon, and are thus free to allow a greater portion of the band to surround each balloon in response to the inflation of the balloon.

Some recorded results are shown in Table I.

TABLE I

| | Systolic | | Cardiac | |
|---|---|---|---|---|
| Heart Rate | Pressure | Ejection | Output | DC Power |
| (bpm) | (mmHg) | Volume (cc) | (L/min) | consumption (Watt) |
| 74 | 128 | 23.7 | 1.75 | 8.8 |
| 75 | 128 | 27 | 2 | 9.8 |
| 83 | 133 | 28.2 | 2.35 | 11 |

In other experiments, performed under slightly varying experimental conditions (e.g., varying lubrication levels), results were obtained as shown in Table II.

TABLE 2

| | | | | | | | DC |
|---|---|---|---|---|---|---|---|
| | | | | | Card. | | Power |
| | | | | Out- | Motor | Avg. | con- |
| Heart | Dias. | Sys. | Ejec. | put | Volt- | cur- | sump- |
| Rate | Pres. | Pres. | Vol. | (cc/ | age | rent | tion |
| (bpm) | (mmHg) | (mmHg) | (cc) | min) | (V) | (mA) | (W) |
| 66.7 | 76.3 | 119.2 | 34.0 | 2267 | 9 | 1220 | 11.0 |
| 75.0 | 71.5 | 114.5 | 24.0 | 1800 | 8.8 | 1666 | 14.7 |
| 71.4 | 71.5 | 104.9 | 25.3 | 1810 | 8.8 | 1533 | 13.5 |
| 75.0 | 71.5 | 119.2 | 23.0 | 1725 | 9 | 794 | 7.1 |
| 83.3 | 76.3 | 133.5 | 28.2 | 2350 | 12 | 915 | 11.0 |
| 74.1 | 76.3 | 128.8 | 23.7 | 1756 | 10 | 877 | 8.8 |
| 75.0 | 76.3 | 128.8 | 27.0 | 2025 | 10 | 976 | 9.8 |

There is therefore provided, in accordance with an embodiment of the present invention, apparatus for compressing at least one chamber of a heart of a patient's body, the apparatus including:

one or more inflatable elements;

a pump in fluid communication with the inflatable elements; and

at least one band having a first one or more first portions and second one or more second portions, the first and second portions alternatingly arranged,
the first one or more first portions and second one or more second portions having respective variable first and second total lengths,
the first one or more first portions adapted to be placed around at least a portion of the heart in mechanical communication with the portion of the heart, and
each of the second portions placed around at least 180 degrees of a periphery of at least one of the inflatable elements, such that the second portions are in mechanical communication with the heart via the first portions, and such that when the inflatable elements are inflated by the pump the first total length decreases by an amount that the second total length increases.

In an embodiment, the first one or more first portions are adapted to be disposed between the heart and the one or more inflatable elements.

In an embodiment, the one or more inflatable elements include respective balloons.

In an embodiment, the one or more inflatable elements include respective piston and cylinder arrangements.

In an embodiment, the pump is adapted to pump a liquid to inflate the inflatable elements.

In an embodiment, the pump is adapted to pump a gas to inflate the inflatable elements.

In an embodiment, for each inflatable element, only one second portion is placed around at least 180 degrees of its periphery.

In an embodiment, the inflatable elements are coupled to the band such that when the first one or more first portions are placed around the portion of the heart, the inflatable elements are symmetrically disposed around the heart.

In an embodiment, the inflatable elements are coupled to the band such that when the first one or more first portions are placed around the portion of the heart, the inflatable elements are asymmetrically disposed around the heart.

In an embodiment, the band includes a tab portion adjacent to one of the inflatable elements, and wherein the band is shaped to define at least one slit thereof adjacent to the one of the inflatable elements, and wherein the tab is adapted to move within the slit responsive to inflation of the one of the inflatable elements.

In an embodiment, the band is adapted to be aligned in parallel with a local muscle fiber direction of the heart.

In an embodiment, the band is adapted to be aligned perpendicularly to a local muscle fiber direction of the heart.

In an embodiment, the band is adapted to be aligned at a divergence of between 20 and 70 degrees from a local muscle fiber direction of the heart.

In an embodiment, the apparatus includes an inner layer, adapted to be disposed between the band and the heart, and at least one hook, adapted to secure the inner layer to the heart.

In an embodiment, the apparatus includes a diastole-supporting mechanism, adapted to store energy from the pump during systole, and to release the energy during diastole in a manner that facilitates application of an outwardly-directed force to an epicardial surface of the heart during diastole.

In an embodiment, the at least one band includes a plurality of bands.

In an embodiment, at least two of the plurality of bands are parallel.

In an embodiment, at least two of the plurality of bands are mutually perpendicular.

In an embodiment, at least two of the plurality of bands diverge by an angle of less than 30 degrees.

In an embodiment, at least two of the plurality of bands diverge by an angle that is between 30 degrees and 45 degrees.

In an embodiment, the apparatus includes an apical-region cover, coupled to the band and adapted to cover a region in a vicinity of an apex of the heart.

In an embodiment, the apical-region cover is adapted to be disposed on the heart such that the vicinity of the apex of the heart does not include the apex.

In an embodiment, the apical-region cover is adapted to cover the apex of the heart.

In an embodiment, the apical-region cover is adapted to passively apply a compressive force to the vicinity of the apex of the heart.

In an embodiment, the apical-region cover is adapted to passively apply a compressive force to the apex of the heart.

In an embodiment, the apical-region cover is adapted to actively apply a compressive force to the vicinity of the apex of the heart.

In an embodiment, at least one of the second portions includes at least one flexible line, which is wrapped at least twice around the periphery of at least one of the inflatable elements.

In an embodiment, the at least one flexible line includes a plurality of flexible lines, each wrapped at least twice around the periphery of the at least one of the inflatable elements.

In an embodiment, at least one of the second portions includes one or more flexible lines, each flexible line adapted to be placed around at least 180 degrees of the periphery of at least one of the inflatable elements.

In an embodiment, the apparatus includes a feedthrough piece shaped to define at least one hole therein, and wherein the one or more flexible lines are adapted to pass through the at least one hole in the feedthrough piece.

In an embodiment, each flexible line passes through a respective one of the at least one hole.

In an embodiment, the one or more flexible lines includes at least 2 lines.

In an embodiment, the plurality of flexible lines includes at least 10 lines.

In an embodiment, the plurality of flexible lines includes at least 25 lines.

In an embodiment, for at least one of the inflatable elements, at least two or more second portions are placed around at least 180 degrees of its periphery.

In an embodiment, the two or more second portions include three or more second portions.

In an embodiment, the apparatus includes a sleeve adapted for placement around the heart, and wherein the band and the inflatable elements are disposed within the sleeve.

In an embodiment, the band is isolated by the sleeve from contact with tissue of the patient's body.

In an embodiment, a total mass of the sleeve including any fluid therein is less than 100 g at all phases of the heart contraction cycle.

In an embodiment, the total mass is less than 50 g at all phases of the heart contraction cycle.

In an embodiment, a total mass of the apparatus is less than 300 g, and wherein the apparatus includes a battery adapted to drive the pump for at least one hour without being recharged from a source outside of the patient's body.

In an embodiment, the battery has a capacity of less than 2 Amp-Hour.

In an embodiment, the battery has a capacity of less than 1.3 Amp-Hour.

In an embodiment, a total volume of the apparatus is less than 300 cc.

In an embodiment, each of the inflatable elements is adapted to increase in volume by at least 0.1 cc in response to the inflation by the pump.

In an embodiment, each of the inflatable elements is adapted to increase in volume by at least 10 cc in response to the inflation by the pump.

In an embodiment, each of the inflatable elements is adapted to increase in volume by less than 80 cc in response to the inflation by the pump.

In an embodiment, each of the inflatable elements is adapted to increase in volume by less than 50 cc in response to the inflation by the pump.

In an embodiment, the one or more inflatable elements include exactly one inflatable element.

In an embodiment, the exactly one inflatable element is adapted to increase in volume by at least 5 cc in response to the inflation by the pump.

In an embodiment, the one or more inflatable elements includes a plurality of inflatable elements.

In an embodiment, the plurality of inflatable elements includes two to three inflatable elements.

In an embodiment, the plurality of inflatable elements includes four to five inflatable elements.

In an embodiment, the plurality of inflatable elements includes greater than six inflatable elements.

In an embodiment, the plurality of inflatable elements includes fewer than 50 elements.

In an embodiment, the plurality of inflatable elements includes fewer than 25 elements.

In an embodiment, a total increase in volume of all of the inflatable elements in response to being inflated by the pump is greater than 5 cc.

In an embodiment, a total increase in volume of all of the inflatable elements in response to being inflated by the pump is greater than 10 cc.

In an embodiment, a total increase in volume of all of the inflatable elements in response to being inflated by the pump is greater than 15 cc.

In an embodiment, a total increase in volume of all of the inflatable elements in response to being inflated by the pump is 25 cc.

In an embodiment, the apparatus is configured such that the decrease of the first total length is at least 8 mm.

In an embodiment, the apparatus is configured such that the decrease of the first total length is at least 40 mm.

In an embodiment, the apparatus is configured such that the decrease of the first total length is less than 150 mm.

In an embodiment, when the inflatable elements are inflated by the pump during a cardiac cycle, a peak reduction in volume of the heart is at least 200% of a total volume of fluid pumped into all of the inflatable elements by the pump during the cardiac cycle.

In an embodiment, when the inflatable elements are inflated by the pump during the cardiac cycle, the peak reduction in volume of the heart is at least 1000% of the total volume of fluid pumped into all of the inflatable elements by the pump during the cardiac cycle.

There is further provided, in accordance with an embodiment of the present invention, apparatus for compressing at least one chamber of a heart of a patients body, the apparatus including:

one or more shape-changing members;

a control unit, coupled to the shape-changing members; and

at least one band having a first one or more first portions and second one or more second portions, the first and second portions alternatingly arranged,
the first one or more first portions and second one or more second portions having respective variable first and second total lengths,
the first one or more first portions adapted to be placed around at least a portion of the heart in mechanical communication with the portion of the heart, and
each of the second portions placed around at least 180 degrees of a periphery of at least one of the shape-changing members, such that the second portions are in mechanical communication with the heart via the first portions, and such that when the shape-changing members are driven by the control unit to change shape, the first total length decreases by an amount that the second total length increases.

In an embodiment, at least one of the shape-changing members includes a hydraulic actuator.

In an embodiment, at least one of the shape-changing members includes an electromechanical actuator.

In an embodiment, the electromechanical actuator includes an electromagnet.

In an embodiment, the electromechanical actuator includes a piezoelectric element.

There is still further provided, in accordance with an embodiment of the present invention, apparatus for compressing at least one chamber of a heart of a patient's body, the apparatus including:

one or more shape-changing members;

a control unit, adapted to drive the shape-changing members to change shape; and

a band, an effective length of the band being adapted to surround a portion of the heart and to shorten responsive to the control unit driving the shape-changing members to change shape, whereby to enhance contraction of the heart.

In an embodiment, the band is adapted to be looped around at least one of the shape-changing members.

In an embodiment, the band is adapted to be looped a plurality of times around at least one of the shape-changing members.

In an embodiment, the band is shaped to define a plurality of discontinuities thereof, and wherein, for each discontinuity, one of the shape-changing members is coupled between an edge of the band on one side of the discontinuity and an edge of the band on another side of the discontinuity.

In an embodiment, the band is shaped to define at least one discontinuity thereof, and wherein one of the shape-changing members is coupled between an edge of the band on one side of the discontinuity and an edge of the band on another side of the discontinuity.

In an embodiment, a plurality of shape-changing members are coupled between the edges of the band.

In an embodiment, at least one of the shape-changing members includes a hydraulic actuator.

In an embodiment, the hydraulic actuator includes a balloon.

In an embodiment, the hydraulic actuator includes a piston and a cylinder.

In an embodiment, the control unit is adapted to drive fluid into the cylinder to cause the effective length of the band to shorten.

In an embodiment, the control unit is adapted to draw fluid out of the cylinder to cause the effective length of the band to shorten.

In an embodiment, at least one of the shape-changing members includes an electromechanical actuator.

There is yet further provided, in accordance with an embodiment of the present invention, apparatus for compressing at least one chamber of a heart, the apparatus including:

one or more inflatable elements;

a pump in fluid communication with the inflatable elements; and

at least one band in mechanical communication with the inflatable elements, a portion of the band adapted to be placed around at least a portion of the heart in mechanical communication with the portion of the heart,

the inflatable elements arranged such that when the inflatable elements are inflated by the pump, the inflatable elements apply more force to the heart via shortening of the portion of the band than via expansion of the inflatable elements against the heart.

There is also provided, in accordance with an embodiment of the present invention, apparatus for compressing at least one chamber of a heart, the apparatus including:

a pump; and

one or more inflatable elements, adapted to be placed around at least a portion of the heart, and in fluid communication with the pump, such that when the inflatable elements are inflated by the pump during a cardiac cycle, a peak reduction in volume of the heart is at least 200% of a total volume of fluid pumped into all of the inflatable elements by the pump during the cardiac cycle.

There is additionally provided, in accordance with an embodiment of the present invention, apparatus for compressing at least one chamber of a heart, the apparatus including:

an implantable compression system; and

a battery sufficient for supporting at least 1 hour of normal operation of the compression system between recharging cycles,

wherein a total mass of the apparatus is less than 300 g.

There is still additionally provided, in accordance with an embodiment of the present invention, apparatus for compressing at least one chamber of a heart, the apparatus including:

an implantable compression system; and

a battery sufficient for supporting at least 1 hour of normal operation of the compression system between recharging cycles;

wherein a total volume of the apparatus is less than 300 cc.

There is yet additionally provided, in accordance with an embodiment of the present invention, apparatus for compressing at least one chamber of a heart, the apparatus including:

an implantable hydraulic compression system,

wherein the system includes a sleeve attached to the heart, and wherein a mass of the sleeve including any fluid therein does not exceed 100 g at any phase of the heart contraction cycle.

In an embodiment, the mass does not exceed 70 g at any phase of the heart contraction cycle.

In an embodiment, the mass does not exceed 50 g at any phase of the heart contraction cycle.

### BRIEF DESCRIPTION OF THE DRAWINGS

FIG. 1 is a schematic illustration of a system for supporting the functioning of the heart of a patient, in accordance with an embodiment of the present invention;

FIG. 2 is a schematic illustration of a cross-section of a balloon-based contraction-enhancement mechanism, for enhancing contraction of the heart, in accordance with an embodiment of the present invention;

FIG. 3 is a schematic illustration of a cross-section of the mechanism of FIG. 2 during the systolic phase of the cardiac cycle, in accordance with an embodiment of the present invention;

FIGS. 4 and 5, are schematic illustrations of a cross-section of an asymmetric balloon-based contraction-enhancement mechanism, for asymmetrically enhancing contraction of the heart, in accordance with an embodiment of the present invention;

FIGS. 6 and 7 are pictorial illustrations of a band-balloon interface for use, in accordance with an embodiment of the present invention;

FIGS. 8A and 8B, are pictorial illustrations of a band-balloon interface, in accordance with another embodiment of the present invention;

FIGS. 9A and 9B are pictorial illustrations of respective arrangements of the components of the system of FIG. 1, in which a sleeve comprises a band-balloon interface and is optionally coupled to an apical-region cover, in accordance with an embodiment of the present invention;

FIG. 10 is a pictorial illustration of a band-balloon interface, in accordance with an embodiment of the present invention;

FIG. 11 is a schematic illustration of a harness for bi-axially decreasing the radius of the heart and increasing ejection of blood therefrom, in accordance with an embodiment of the present invention;

FIG. 12 is a schematic illustration of an interface between a hydraulically-actuated cushion and a linear element wrapped around the cushion, in accordance with an embodiment of the present invention;

FIG. 13 is a schematic illustration of a cross-section of a piston-based contraction-enhancement mechanism, for enhancing contraction of the heart, in accordance with an embodiment of the present invention;

FIG. 14 is a schematic illustration of a piston arrangement for enhancing contraction of the heart, in accordance with an embodiment of the present invention;

FIG. 15 is a schematic illustration of a piston arrangement, for enhancing contraction of the heart, in accordance with another embodiment of the present invention;

FIG. 16 is a schematic illustration of an amplification arrangement, for enhancing contraction of the heart, in accordance with an embodiment of the present invention;

FIG. 17 is a schematic illustration of a band-cylinder interface, for enhancing contraction of the heart, in accordance with an embodiment of the present invention;

FIG. 18 is a schematic illustration of an attachment system for attaching to the heart any of the apparatus described hereinabove, in accordance with an embodiment of the present invention;

FIG. 19 is a schematic, cross-sectional illustration of an attachment mechanism for attaching a sleeve inner wall to the myocardium of the heart, in accordance with an embodiment of the present invention;

FIGS. 20 and 21 are schematic illustrations of diastolic and systolic phases, respectively, of a diastole-support mechanism, in accordance with an embodiment of the present invention;

FIG. 22 is a schematic illustration of a diastole-support mechanism, in accordance with an embodiment of the present invention; and

FIGS. 23A to 46H are schematic illustrations of cardiac apparatus, in accordance with respective embodiments of the present invention.

### DETAILED DESCRIPTION OF EMBODIMENTS

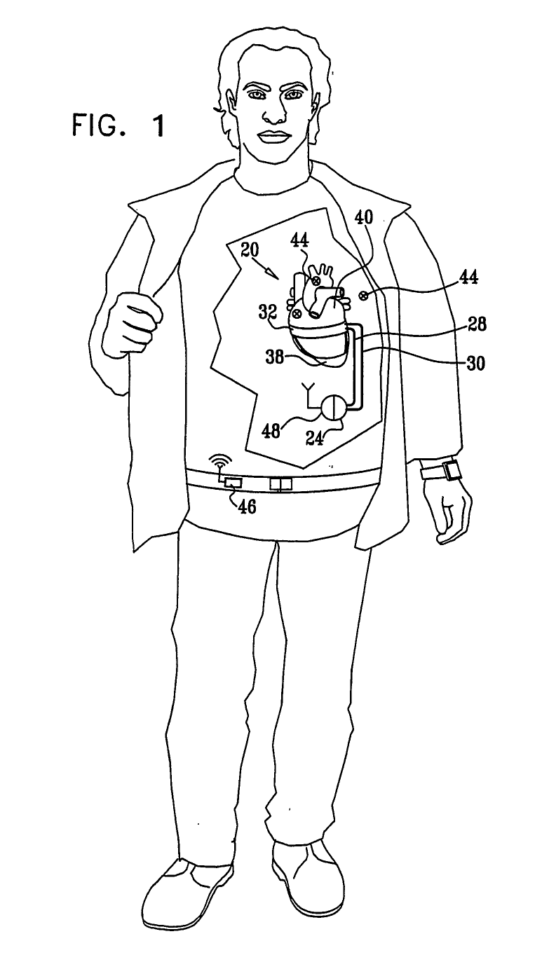

FIG. 1 is a schematic illustration of a system 20 for supporting the functioning of a heart 40 of a patient, in accordance with an embodiment of the present invention. System 20 comprises a sleeve 32, which surrounds one or more chambers of heart 40, and a control unit 24, which actuates the sleeve to apply a compressive force to heart 40. Control unit 24 is in operational communication with sleeve 32 via one or more electrical leads 28 and/or one or more tubes 30. For example, control unit 24 may comprise a pump that drives a fluid (i.e., a liquid or a gas) through tubes 30, into or out of sleeve 32, whereby to cause contraction of the sleeve around the heart and a resultant enhancement of cardiac output. Alternatively or additionally, control unit 24 drives a current through leads 28, whereby to cause contraction of the sleeve around the heart and a resultant enhancement of cardiac output. For some applications, control unit 24 receives signals, wirelessly or through leads 28, from one or more sensors 44 coupled to a heart-contacting surface of sleeve 32 or to other sites of the patient.

An internal power supply 48 of control unit 24 is typically in near-continuous wireless communication with an external power supply 46. Internal power supply 48 typically comprises a rechargeable battery whose charge is maintained by inductive transfer of power from external power supply 46. When internal power supply 48 is not receiving power from external power supply 46 (e.g., for periods of over an hour), the internal power supply enhances the contractions of heart 40 using the energy stored in the battery.

For some applications, sleeve 32 is placed so as to generally cover two chambers (as in FIG. 1). Alternatively, the sleeve covers three or four chambers of the heart. As appropriate, an apical-region cover 38 is coupled to sleeve 32, and covers or surrounds the apex of heart 40.

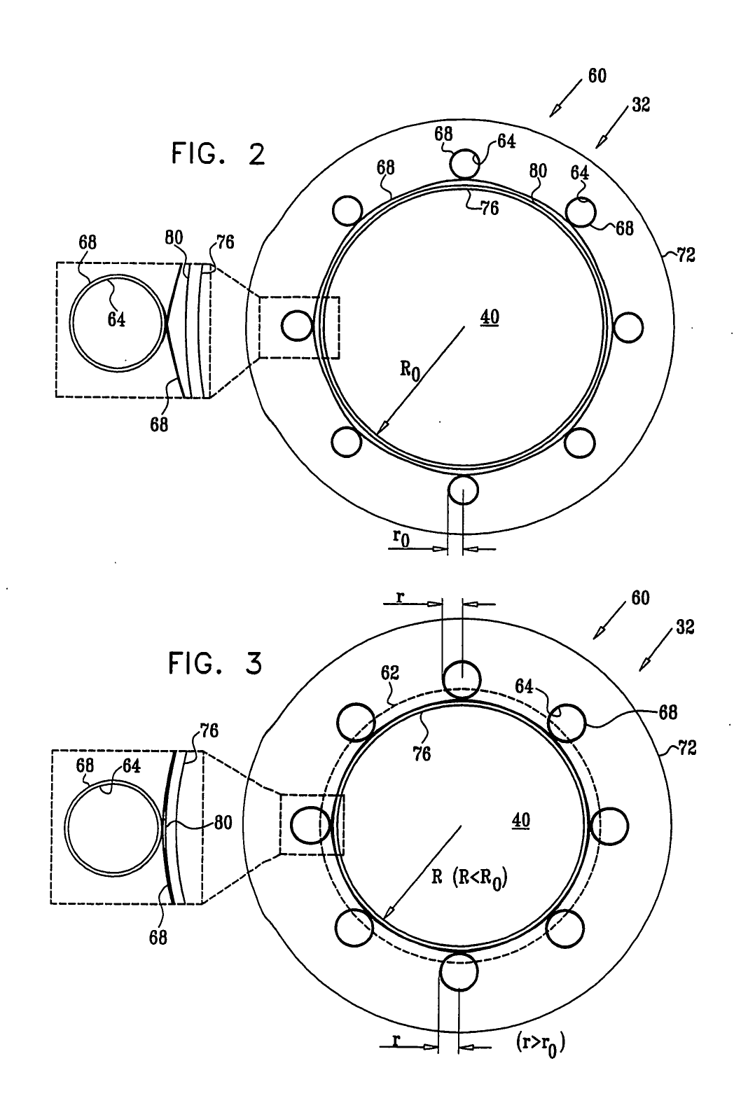

FIG. 2 is a schematic illustration of a cross-section of a balloon-based contraction-enhancement mechanism 60, for enhancing contraction of heart 40, in accordance with an embodiment of the present invention. FIG. 2 shows heart 40 during the diastolic phase of the cardiac cycle.

Mechanism 60 is disposed within sleeve 32, between an outer wall 72 of the sleeve and an inner wall 80 of the sleeve. Inner wall 80 typically lies directly against the epicardium 76 of heart 40. Mechanism 60 comprises one or more inflatable elements such as balloons 64, and a band 68 looped around each balloon. For simplicity of the figures, connections are generally not shown between control unit 24 and balloons 64 in FIG. 2, or between control unit 24 and other devices in the other figures.

FIG. 3 is a schematic illustration of a cross-section of mechanism 60 during the systolic phase of the cardiac cycle, in accordance with an embodiment of the present invention. To facilitate a comparison of FIG. 2 and FIG. 3, a dashed line 62 in FIG. 3 shows the heart as it had been during diastole, in comparison to the smaller radius that the heart attains during systolic contraction.

In order to support systolic contraction, control unit 24 (FIG. 1) drives a fluid into each balloon 64, thereby inflating the balloons from an initial radius r0 to a final radius r. The radius of each balloon therefore changes by a value dr=r−r0. In response to the increased size of each balloon 64, band 68 (which is looped around each of the balloons) is forced to surround larger balloons, and, since the band is of generally fixed length, a smaller length of the band is available to be in contact with inner wall 80 of sleeve 32. The band, as shown, is constrained to continue to surround heart 40 regardless of the portion of the cardiac cycle. Therefore, when the portion of the band in contact with inner wall 80 decreases in length, the perimeter of the heart adjacent to band 68 decreases, as well. The decrease in perimeter corresponds to a decrease in radius dR of the heart from an initial (diastolic) radius R0 to a final (systolic) radius R. This decrease in radius, in turn, increases the ejection of blood from heart 40.

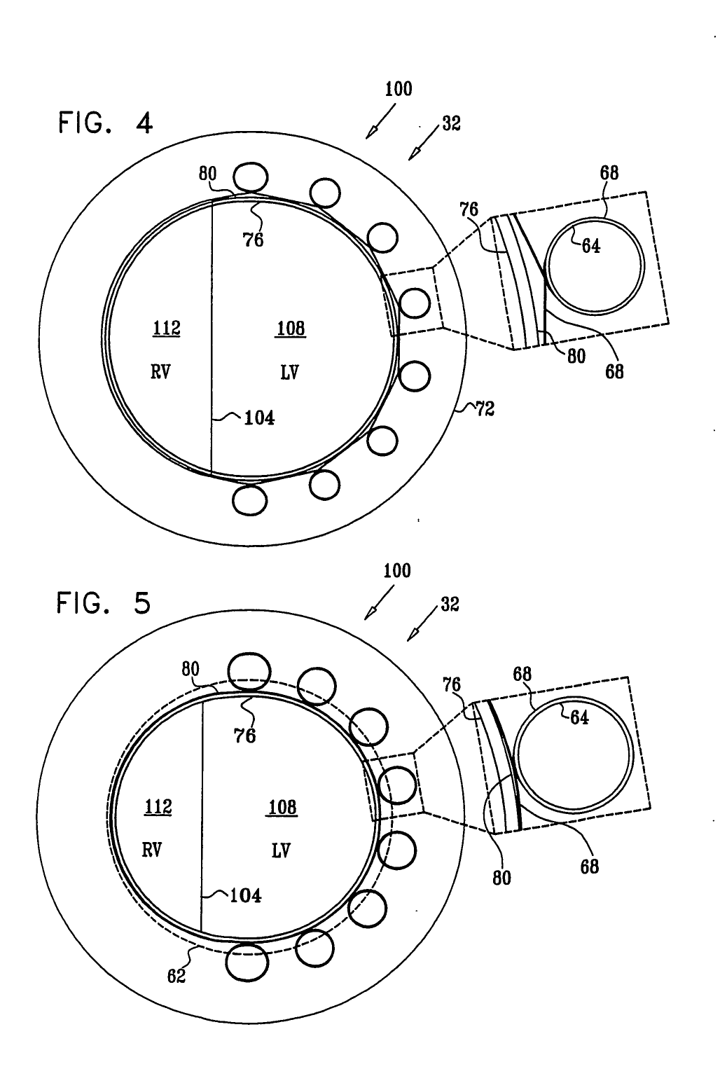

Reference is now made to FIGS. 4 and 5, which are schematic illustrations of a cross-section of an asymmetric balloon-based contraction-enhancement mechanism 100, for asymmetrically enhancing contraction of heart 40, in accordance with an embodiment of the present invention. FIG. 4 shows heart 40 during the diastolic phase of the cardiac cycle, and FIG. 5 shows heart 40 during the systolic phase of the cardiac cycle. Asymmetric mechanism 100 is generally similar to mechanism 60 described with reference to FIGS. 2 and 3, except for differences as noted hereinbelow.

In mechanism 100, balloons 64 are distributed asymmetrically around heart 40. For example, as shown in FIG. 4, eight balloons 64 may be distributed so as to predominantly be adjacent to the left ventricle 108 of heart 40 (the “active” area of force application), and to be adjacent to right ventricle 112 (the “non-active” area of force application) only to a lesser extent, or not at all. A line 104 between ventricles 108 and 112 symbolically represents the interventricular septum. During systole, balloons 64 inflate (FIG. 5), thereby shortening the length of band 68 that is in contact with inner wall 80 of sleeve 32. Because of the positioning of the balloons predominantly over left ventricle 108, the effect of increased ejection of blood is greater from left ventricle 108 than from right ventricle 112. As appropriate, mechanical coupling between band 68 and inner wall 80, and/or different elastic properties of respective portions of band 68 are set so as to facilitate a desired asymmetric distribution of the applied contraction-enhancing force.

It is to be appreciated that FIGS. 4 and 5 show asymmetric mechanism 100 having an asymmetry with respect to the two ventricles, by way of illustration and not limitation. In other embodiments (not shown), asymmetric mechanism 100 is applied to the heart so as to asymmetrically enhance contraction of other chambers.

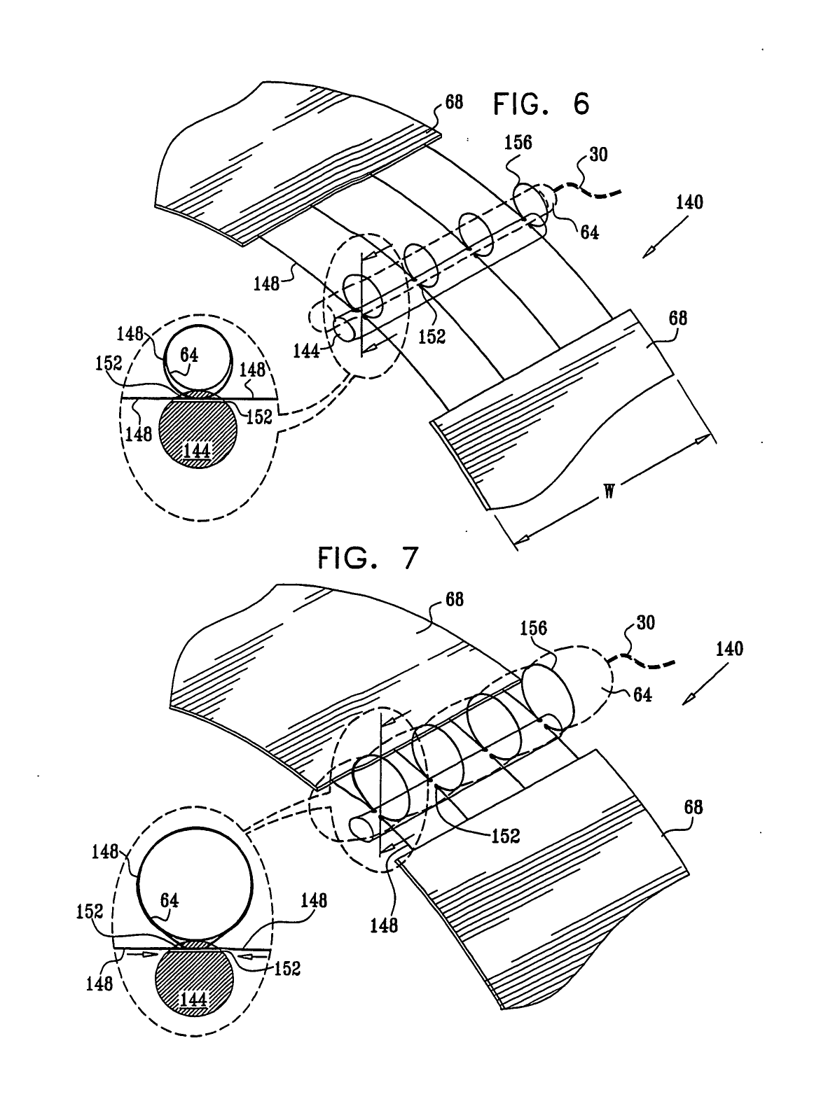

FIG. 6 is a pictorial illustration of a band-balloon interface 140 for use, for example, with mechanisms 60 or 100, in accordance with an embodiment of the present invention. Band-balloon interface 140 is shown in FIG. 6 during the diastolic phase of the cardiac cycle. Interface 140 comprises a feedthrough piece 144 and one or more flexible lines 148. Each flexible line 148 is attached at either end to respective portions of band 68, and passes through holes 152 in feedthrough piece 144. During assembly of band-balloon interface 140, respective loops 156 are formed by passing each flexible line 148 through the feedthrough piece. One of balloons 64 is placed within the loops 156 formed by flexible lines 148. Alternatively, more than one balloon 64 is placed within loops 156.

Typically, but not necessarily, band-balloon interface 140 comprises about 1 to about 16 lines 148, e.g., about 14 lines. (Four lines 148 are shown in the figure.) Each line is typically between about 10 mm and about 60 mm long, e.g., about 50 mm long. For some applications, the lines themselves comprise a textile, expanded polytetrafluoroethylene (ePTFE) wire, polyvinylidene fluoride (PVDF) wire, polypropylene, polyethylene, nylon, nylon-66, or cotton, and can typically be viewed as being substantially inelastic under the forces present during cyclic use of band-balloon interface 140. The outer surface of balloon 64 typically comprises a polymer or a metal, is coated with a lubricant, and/or is covered with a protective covering, in order to reduce friction between the balloon and flexible lines 148.

Feedthrough piece 144 typically is of a generally longitudinal shape (e.g., a cylinder), as shown in FIG. 6. Alternatively, feedthrough piece 144 is of a different shape, e.g., a generally planar shape, with holes 152 passing therethrough. The width (W) of band 68 is typically between about 1 cm and about 10 cm, and typically, but not necessarily, corresponds to the length of feedthrough piece 144.

FIG. 7 is a pictorial illustration of band-balloon interface 140 during systole, in accordance with an embodiment of the present invention. During inflation of balloon 64, a portion of each flexible line 148 is pulled into feedthrough piece 144 in order to provide the added length of line 148 for loop 156 due to the inflation of the balloon. The pulling of flexible lines 148 into or through feedthrough piece 144, in turn, pulls the two ends of band 68 (FIG. 7) closer together. In this manner, the effective total perimeter of band 68 in contact with inner wall 80 of sleeve 32 is reduced, and a compressive force is thereby applied to heart 40, increasing the ejection of blood therefrom.

It is to be appreciated that in some embodiments (not shown), a discrete feedthrough piece is not provided, and at least some of the functionality of the feedthrough piece is provided instead by the arrangement of flexible lines 148 and/or band 68.

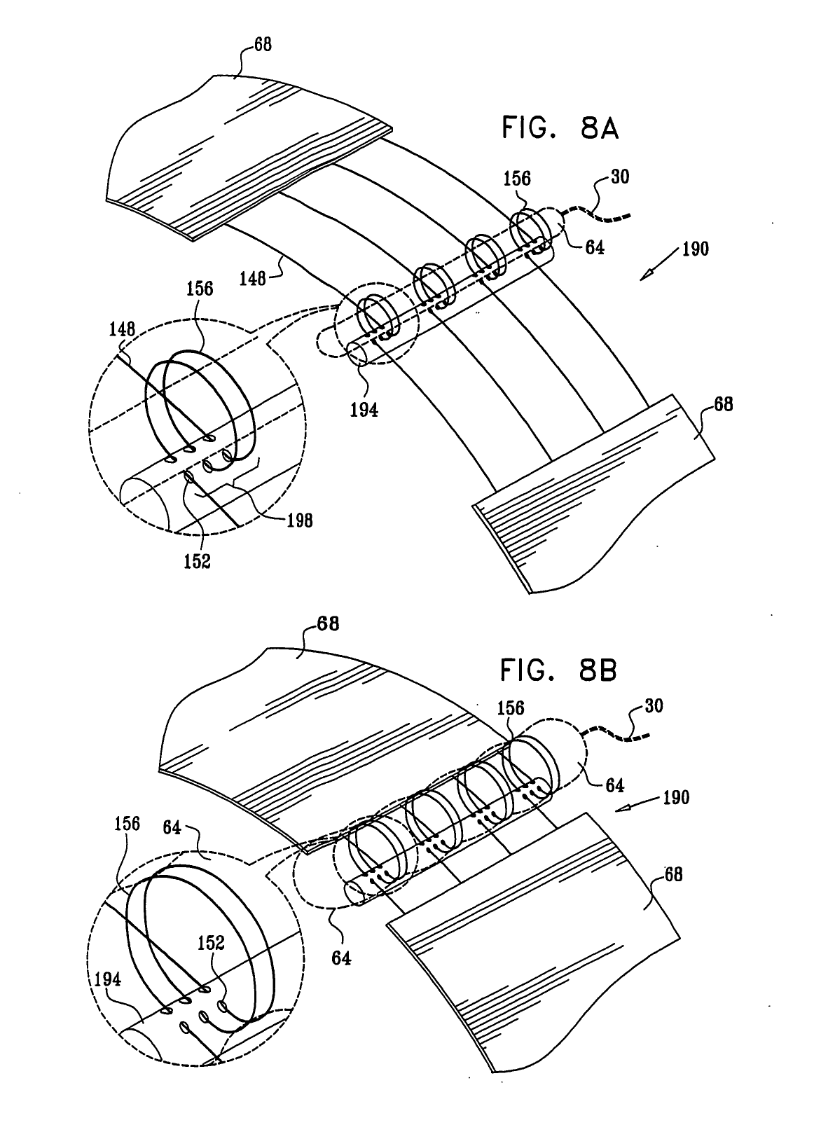

Reference is now made to FIGS. 8A and 8B, which are pictorial illustrations of a band-balloon interface 190 for use, for example, with mechanisms 60 or 100, in accordance with an embodiment of the present invention. Band-balloon interface 190 is shown during diastole in FIG. 8A, and during systole in FIG. 8B. Band-balloon interface 190 is generally similar to band-balloon interface 140 described with reference to FIGS. 6 and 7, except for differences as noted hereinbelow.

Band-balloon interface 190 typically comprises a feedthrough piece 194, which has a plurality of holes 152 through which one or more flexible lines 148 pass. Unlike in band-balloon interface 140, however, each of the flexible lines loops a plurality of times around balloon 64. FIG. 8A, for example, shows the flexible lines looping twice around balloon 64. For some applications, the flexible lines loop three, four, or more times around the balloon. The use of N>1 loops in each flexible line 148 typically provides further enhancement of cardiac output, compared to the use of a single loop. In general, if a single loop yields a change dR in heart radius, then N loops yield a change of N*dR, ceteris paribus.

For some applications, the multiple loops pass through respective holes 152 in feedthrough piece 190. In an embodiment, holes 152 are arranged in sets 198 of holes (FIG. 8A), one set for each flexible line. In another embodiment (e.g., as shown in FIG. 6), the holes are not arranged into separate sets. In yet another embodiment, the multiple loops surround balloon 64, without a discrete feedthrough piece to support the loops.

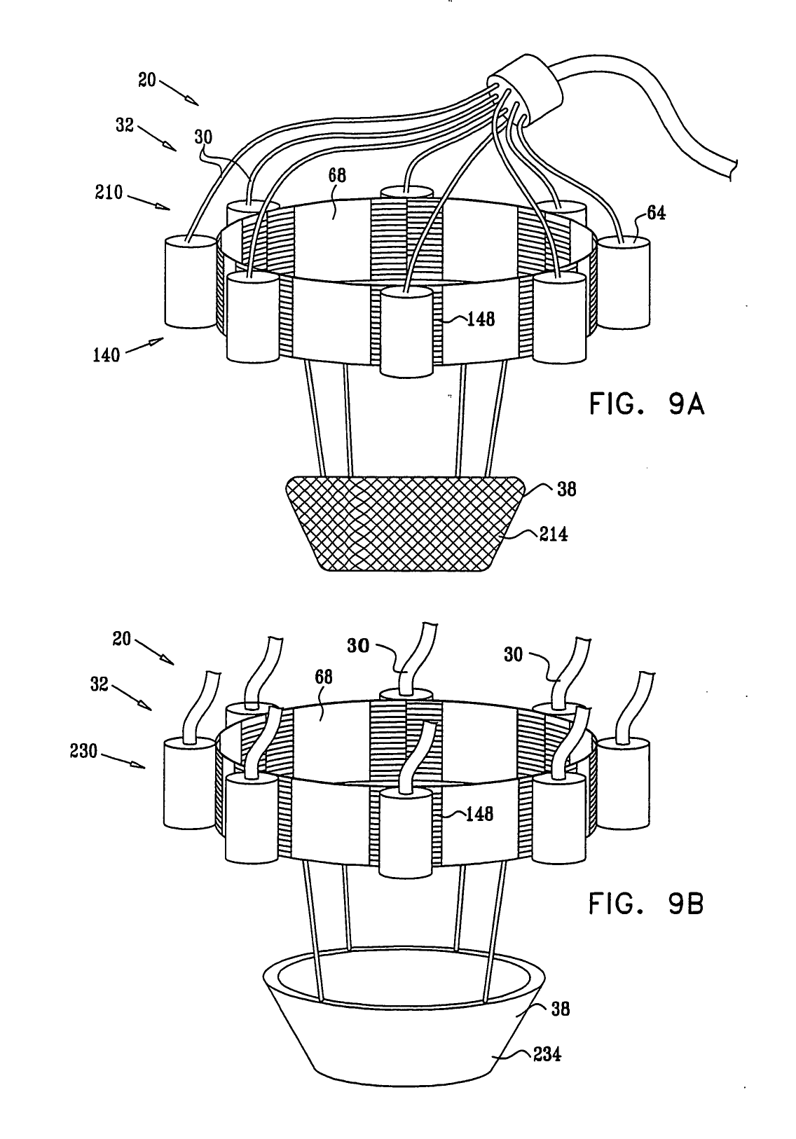

FIG. 9A is a pictorial illustration of an arrangement 210 of the components of system 20 (FIG. 1), in which sleeve 32 comprises band-balloon interface 140 and is optionally coupled to apical-region cover 38, in accordance with an embodiment of the present invention. In this embodiment, apical-region cover 38 comprises an apex cover 214. Cover 214 typically comprises a stiff or a flexible material, e.g., a mesh and/or an elastic material, and is typically a passive component of system 20, generally serving to maintain the proper positioning of system 20 with respect to heart 40. For some applications, however, apex cover 214 is an active component of system 20, cyclically applying a compressive force to the heart. In this case, apex cover 214 is typically driven by control unit 24, electrically or mechanically, in temporal coordination with the actuation by control unit 24 of sleeve 32 to apply compressive forces to heart 40.

FIG. 9B is a pictorial illustration of an arrangement 230 of the components of system 20 (FIG. 1), in which sleeve 32 comprises band-balloon interface 140 and is optionally coupled to apical-region cover 38, in accordance with an embodiment of the present invention. In this embodiment, apical-region cover 38 comprises a mechanical stabilizer 234, e.g., a ring-shaped stabilizer, that substantially does not cover the apex of heart 40. Thus, stabilizer 234 is open at its base (opening not shown). Mechanical stabilizer 234 typically comprises a polymer, metal, or textile, and is typically a passive component of system 20, generally serving to maintain the proper positioning of system 20 with respect to heart 40.

It is noted that although FIGS. 9A and 9B show flexible lines 148 surrounding essentially the entire length of each balloon 64 (see exploded view in FIG. 2 for more detail), the scope of the present invention includes having lines 148 surround a smaller portion of the length of each balloon, e.g., about 10% to about 40% of the length of the balloon, or about 40% to about 85% of the length of the balloon.

FIG. 10 is a pictorial illustration of a band-balloon interface 250, for use, for example, with mechanisms 60 or 100, in accordance with an embodiment of the present invention. Band-balloon interface 250 is shaped to define a tab 258 and a slit 254. Tab 258 wraps around balloon 64 and passes through slit 254, prior to merging with band 68. In an embodiment, tab 258 is a shaped portion of band 68.

As balloon 64 inflates, the two ends of band 68 adjacent to balloon 64 are pulled together, thereby decreasing the perimeter of heart 40 and increasing ejection of blood therefrom. Although FIG. 10 shows a single band-balloon interface coupled to balloon 64, for some applications, a plurality of band-balloon interfaces are coupled to the balloon, at respective positions along the length of the balloon.

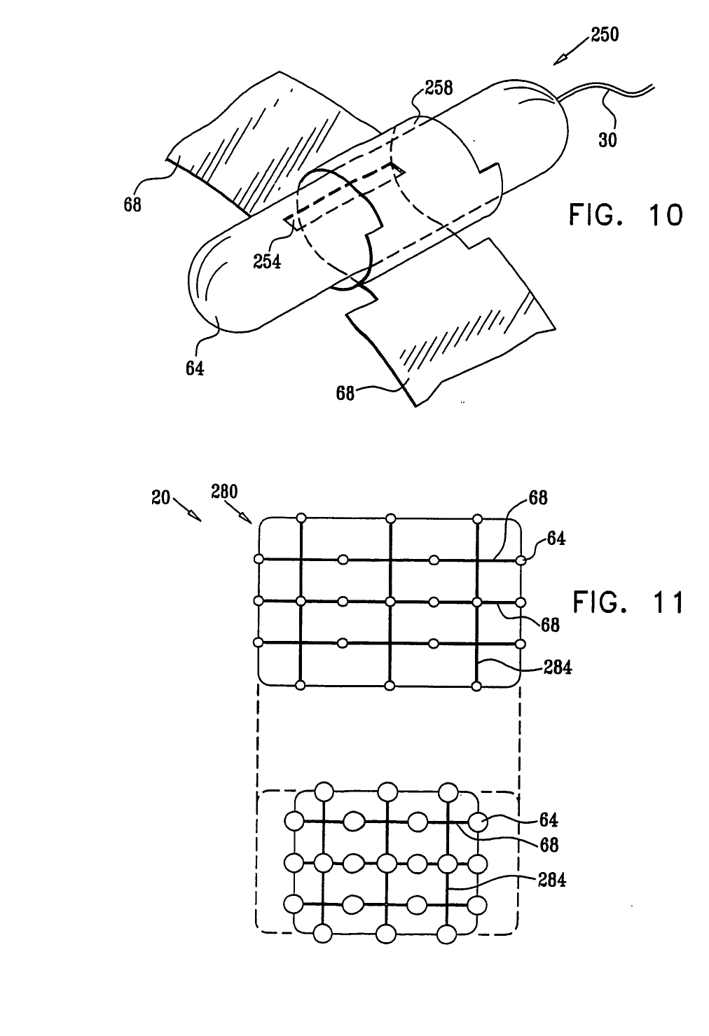

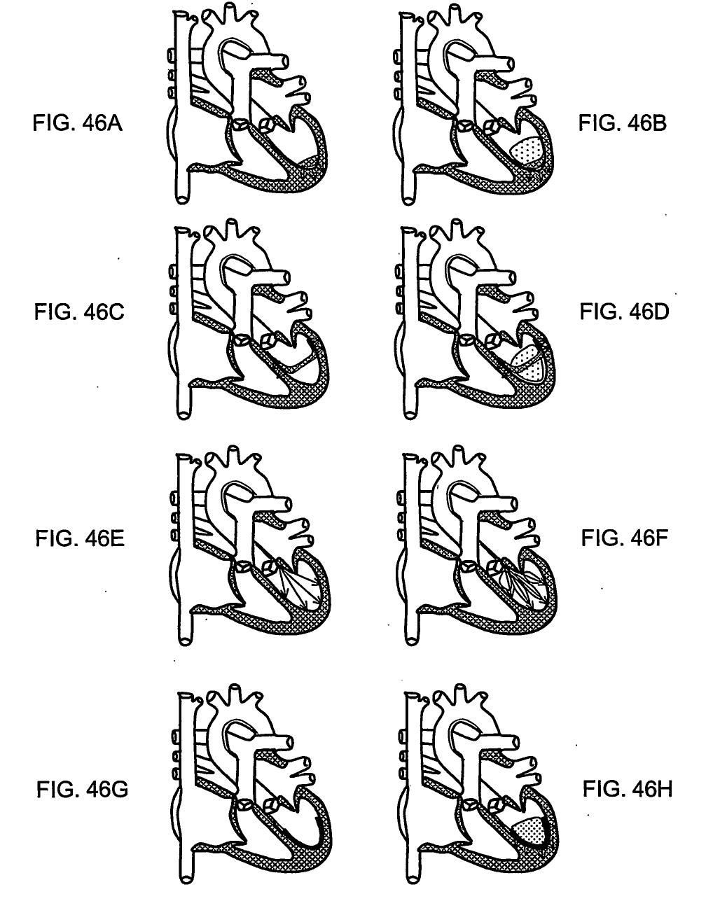

FIG. 11 is a schematic illustration of a harness 280 for bi-axially decreasing the radius of heart 40 and increasing ejection of blood therefrom, in accordance with an embodiment of the present invention. As described in the above-cited U.S. Provisional Patent Application 60/511,548 to Kornowski and Bar, which is incorporated herein by reference, harness 280 comprises (a) widthwise strips, typically a plurality of horizontally-oriented bands 68, and (b) lengthwise strips, typically a plurality of vertically-oriented bands 284. Harness 280 is thereby divided into multiple sections. Bands 68 and 284 are formed into loops (e.g., as described hereinabove), and a balloon 64 is placed within each loop. Some of balloons 64 are within a loop formed by only a single one of the bands (e.g., band 68, as shown), while others of balloons 64 are within loops formed by both bands. For some applications, balloons coupled to bands 68 are inflated at the same time as balloons coupled to bands 284 (FIG. 11), while for other applications, the two sets of balloons are independently controlled (FIG. 45B).

Inflation of balloons 68 enlarges their cross-sectional circumferences, causing the portions of each of the strips in contact with heart 40 to be effectively reduced in length (because the available length is pulled around the balloons). This effective reduction in length enhances contraction of the heart.

As appropriate based on the physiology or pathology of an individual patient's heart, band 68 or band 284 may be oriented to be mutually perpendicular, or at a non-orthogonal angle with respect to each other. For example, they may be separated by an angle of between about 5 and about 20 degrees, or between about 20 and about 45 degrees. Alternatively or additionally, one or both of the bands is substantially aligned with the orientation of heart muscle fibers adjacent to the band. For some applications, one or both bands are oriented at an angle substantially diverging from the local heart muscle fiber orientation, e.g., by greater than 10 degrees, by greater than 30 degrees, or by being nearly orthogonal to the local fiber orientation.

FIG. 12 is a schematic illustration of an interface between a hydraulically-actuated cushion 300, and a linear element 304 wrapped around the cushion, in accordance with an embodiment of the present invention. As described in the above-cited U.S. Provisional Patent Application 60/511,548 to Kornowski and Bar, which is incorporated herein by reference, linear element 304 may comprise a spring. Alternatively or additionally, linear element 304 comprises band 68 and/or one of lines 148 described hereinabove. (It is noted that band 68 and lines 148 may be identical, or may be separate components.) As appropriate for any given application, linear element 304 may have spring properties (i.e., be elastic), or linear element 304 may be generally inelastic.

For some applications, hydraulically-actuated cushion 300 comprises one of balloons 64. Application of hydraulic or other forces to cushion 300 inflates the cushion, and pulls a greater length of linear element 304 to surround the cushion. In some embodiments of the present invention, the interface between cushion 300 and linear element 304 is utilized in combination with one or more of the embodiments described hereinabove with reference to FIGS. 1-11.

FIG. 13 is a schematic illustration of a cross-section of a piston-based contraction-enhancement mechanism 320, for enhancing contraction of heart 40, in accordance with an embodiment of the present invention. Mechanism 320 comprises one or more piston/cylinder arrangements 322, each typically comprising a cylinder 324 and a piston 328 slidably coupled thereto. Each piston arrangement 322 is coupled to band 68 such that removal of fluid from cylinder 324 draws piston 328 further into the cylinder, thereby shortening the effective length of band 68 around the heart and enhancing the ejection of blood therefrom.

In an embodiment, about 0.5 cc to about 2 cc of fluid enters and leaves each piston arrangement 322 during each cardiac cycle.

In an embodiment, a total of about 4 to about 10 cc of fluid enters and leaves all of the piston arrangements 322 in mechanism 320 during each cardiac cycle.

In an embodiment, mechanism 320 comprises between about 1 and about 10 piston arrangements 322.

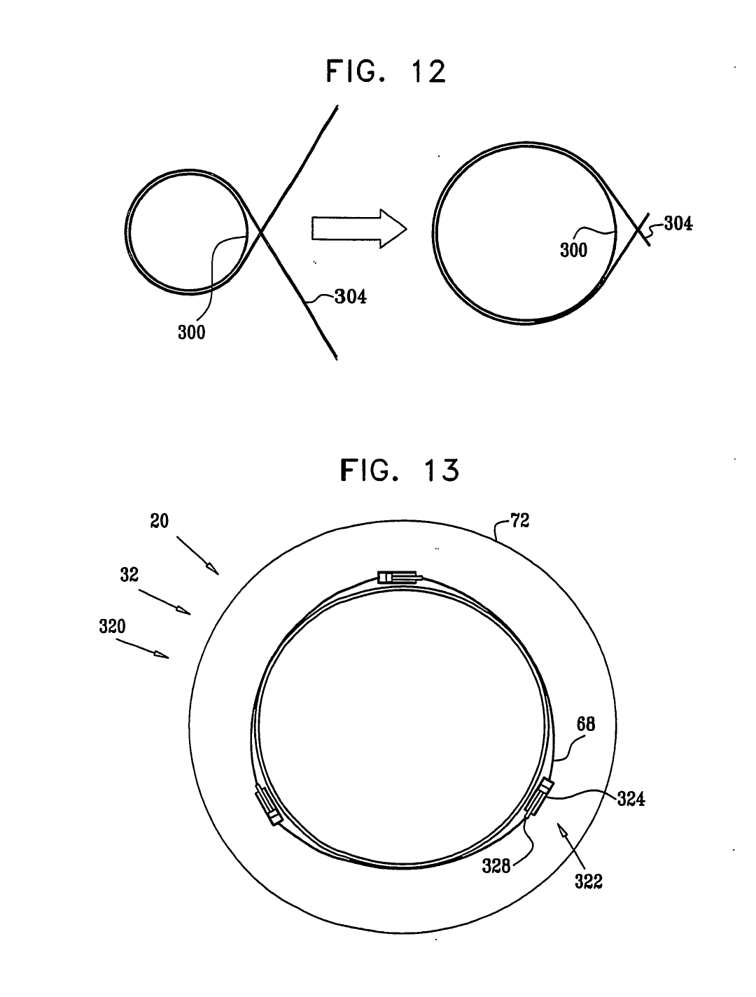

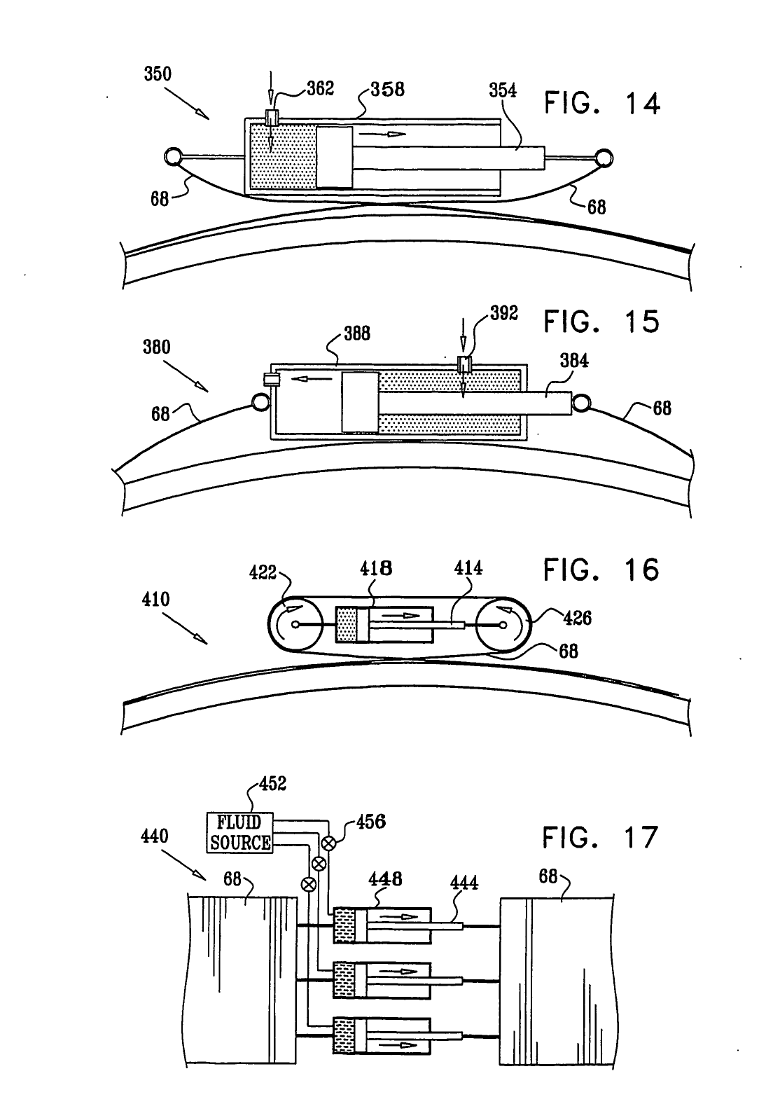

FIG. 14 is a schematic illustration of a piston arrangement 350, for enhancing contraction of heart 40, in accordance with an embodiment of the present invention. Piston arrangement 350 comprises a cylinder 358, a piston 354 slidably coupled thereto, and a port 362 in the cylinder, opposite the rod side of the cylinder. When control unit 24 (FIG. 1) forces fluid through port 362 into cylinder 358, the piston is forced out of the cylinder, thereby shortening the effective length of band 68 that surrounds the heart. This shortening enhances ejection of blood from the heart.

FIG. 15 is a schematic illustration of a piston arrangement 380, for enhancing contraction of heart 40, in accordance with an embodiment of the present invention. Piston arrangement 380 comprises a cylinder 388, a piston 384 slidably coupled thereto, and a port 392 in the cylinder, on the rod side of the cylinder. When control unit 24 (FIG. 1) forces fluid through port 392 into cylinder 388, the piston is forced into the cylinder, thereby shortening the effective length of band 68 that surrounds the heart. This shortening enhances ejection of blood from the heart.

It is noted that in the unlikely event of a malfunction of system 20 (e.g., if internal power supply 48 is uncharged, or if a fluid leak develops anywhere in system 20), then no fluid is forced into cylinder 388. In this case, the total mass of relatively-empty piston arrangement 380 is minimized, and the inertial effect of piston arrangement 380 on heart 40 is minimized. Typically, but not necessarily, the mass of piston arrangement 380, when empty, is less than 50 g. For example, the mass may be less than 20 g. A typical value of the mass of piston arrangement 380, when empty, is about 5 g.

FIG. 16 is a schematic illustration of an amplification arrangement 410, for enhancing contraction of heart 40, in accordance with an embodiment of the present invention. Amplification arrangement 410 comprises (a) a cylinder 418, (b) a piston 414 slidably coupled thereto, and (c) a wheel 422 coupled to the cylinder and/or a wheel 426 coupled to the piston. Band 68 is looped around wheels 422 and/or 426 (typically both wheels), such that when control unit 24 (FIG. 1) forces fluid into cylinder 418, the piston is forced out of the cylinder, thereby shortening the effective length of band 68 that surrounds the heart. This shortening enhances ejection of blood from the heart.

It is noted that in the unlikely event of a malfunction of system 20 (e.g., if internal power supply 48 is uncharged, or if a fluid leak develops anywhere in system 20), then no fluid is forced into cylinder 418. In this case, the total mass of relatively-empty amplification arrangement 410 is minimized, and the inertial effect of amplification arrangement 410 on heart 40 is minimized. Typically, but not necessarily, the mass of amplification arrangement 410 (including the empty cylinder and both wheels) is less than 50 g. For example, the mass may be less than 20 g. A typical value of the mass of amplification arrangement 410, when empty, is about 5 g.

It is further noted that although FIG. 16 shows a piston and cylinder coupled to wheels 422 and 426, this is by way of illustration and not limitation. In other embodiments, other shape-changing members are used, in addition to or instead of the piston and cylinder. For example, such a shape-changing member may comprise a hydraulic actuator (such as a balloon), or an electromechanical actuator (such as an electromagnet or a piezoelectric actuator).

It is still further noted that whereas FIG. 16 shows band 68 looped once around wheels 422 and 426, the scope of the present invention includes looping the band around the wheels a plurality of times. In one embodiment, such multiple loops are used to achieve mechanical amplification of a mechanical input (e.g., a 1 mm displacement of a high-force-low-displacement actuator, such as a piezoelectric actuator) to a larger mechanical output (e.g., a 4-15 mm reduction in effective size of band 68). For some single- or multiple-loop embodiments, the force generated by the actuator (whether hydraulic or electromechanical) is greater than 30 N (for example, greater than 100 N), and is applied at this magnitude during a displacement of 1-3 mm, or during a displacement of greater than 3 mm.

FIG. 17 is a schematic illustration of a band-cylinder interface 440, for enhancing contraction of heart 40, in accordance with an embodiment of the present invention. Band-cylinder interface 440 comprises a plurality of pistons 444 and cylinders 448, coupled in parallel between segments of band 68. A fluid source 452, typically incorporated within control unit 24, withdraws fluid from cylinders 448 in order to enhance systolic contraction of heart 40, and, for some applications, drives fluid into the cylinders in order to facilitate diastolic filling of the heart.

For some applications, band-cylinder interface 440 comprises one or more flow regulators 456 coupled between fluid source 452 and the cylinders. FIG. 17 shows a particular embodiment in which each cylinders has a respective flow regulator 456 coupled thereto. In another embodiment, one or more of flow regulators 456 are disposed so as to allow fluid flow between the cylinders. For some applications, the shape of the inside of each cylinder 448 defines flow regulator characteristics thereof, or the cylinder comprises a discrete flow regulator 456.

In an embodiment of the present invention, flow regulators 456 are substantially identical in operation. Typically, this facilitates a generally even, parallel, synchronous application of force across the width of band 68.

In another embodiment, flow regulators 456 do not operate identically. Alternatively or additionally, cylinders 448 do not operate identically in response to an identical mechanical input. For example, the cylinders may have different physical dimensions. In an embodiment of the present invention, each flow regulator has its own respective flow regulator pressure threshold, below or above which the flow regulator inhibits fluid flow therethrough. Alternatively or additionally, the flow regulators differ in another mechanical characteristic thereof (e.g., each flow regulator may be embodied as a diameter constriction of a tube leading to the respective cylinder).

In an embodiment, the flow regulators are electronically coupled to control unit 24, and are actuated by the control unit to open or close. For some applications, the control unit modulates its own behavior or the behavior of the flow regulators in response to an external command (e.g., transmitted inductively to the control unit), or in response to a sensed physiological parameter (e.g., heart rate).

For some applications, non-identical mechanical behavior of flow regulators 456 and/or cylinders 448 and/or pistons 444 facilitates application of a contraction wave across the width of band 68 (e.g., top to bottom, or bottom to top). For example, such a wave may be timed to closely lead, closely follow, or coincide with the passage of a local, physiological contraction wave of the heart, in the vicinity of band-cylinder interface 440. Alternatively or additionally, a plurality of band-cylinder interfaces 440 disposed around the heart (e.g., at respective positions along band 68) are activated to enhance a contraction wave of the heart. In this case, the contraction enhanced by each interface 440 is typically timed to closely lead, closely follow, or coincide with the passage of a local, physiological contraction wave of the heart.

FIG. 17 shows rods extending from cylinders 448 and pistons 444. These rods reach band 68, in order to pull the band. For some applications, a stiff edge region of band 68 (not specifically delineated in the figure) is disposed at the ends of band 68, where it is coupled to the rods extending from the cylinders and pistons, in order to facilitate controlled pulling of the band. In an embodiment, a mechanical property of the edge region varies across the length of the edge region, in order to shape the response of the band to the movement of pistons 444 and/or cylinders 448. Alternatively or additionally, a length of one of the rods differs from a length of another one of the rods by at least 10%, in order to shape the response of the band to the movement of pistons 444 and/or cylinders 448. Alternatively or additionally, a length of one of the pistons differs from a length of another one of the pistons by at least 10%, in order to shape the response of the band to the movement of pistons 444 and/or cylinders 448.

FIG. 17 shows three cylinders. In an embodiment (configuration not shown), band-cylinder interface 440 comprises one cylinder 448 (e.g., the top cylinder in the figure). The rod connected to the cylinder and the rod connected to its piston are coupled to a V-shaped hinge, whereby movement of the piston within the cylinder opens and closes the hinge. The joint of the hinge may be, for example, where the lower cylinder in FIG. 17 is shown (the lower cylinder being typically absent in this embodiment). Rods extend from the arms of the V-shaped hinge to respective portions across the width of the band (e.g., to the three rod-band connection sites shown in FIG. 17). The band is pulled to a different extent (or with different timing) depending on whether it is being primarily acted upon by (a) the portion of the V-shaped hinge near the hinge's joint, or (b) the ends of the arms of the V-shaped hinge.

It is noted that the mechanical behaviors attained by the hydraulic components shown in FIG. 17 may also be attained, in some embodiments, using electromechanical components.

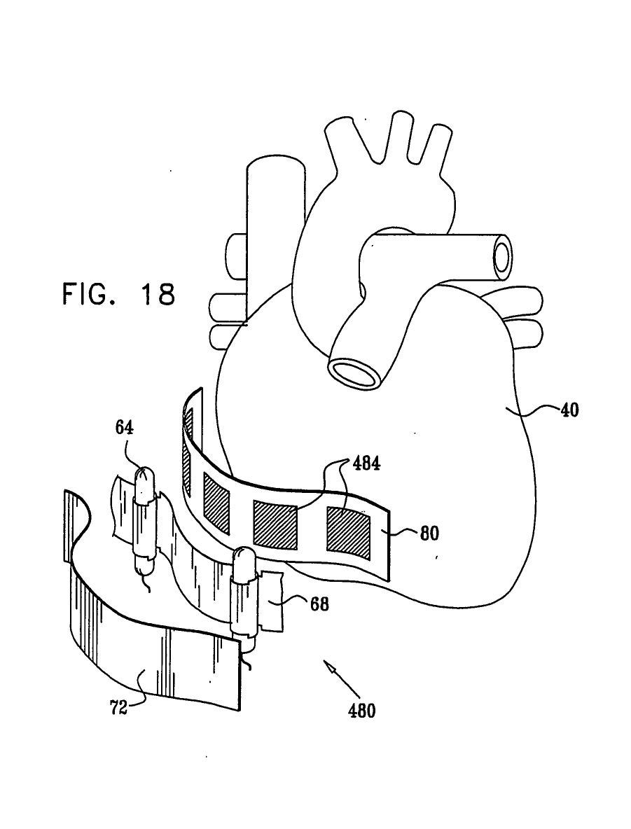

FIG. 18 is a schematic illustration of an attachment system 480 for attaching to the heart any of the apparatus described hereinabove, in accordance with an embodiment of the present invention. Portions of outer wall 72 and inner wall 80 are shown surrounding belt 68. Belt 68 is shown as being configured in accordance with the embodiment described hereinabove with reference to FIG. 10, although other apparatus described herein for enhancing contraction of the heart may be used, as well.

Inner wall 80 typically comprises a plurality of discrete heart-interface portions 484 (as shown). Alternatively, inner wall 80 comprises a single, distributed heart-interface portion 484. For some applications, heart-interface portions 484 are configured in order to strengthen the bonding between inner wall 80 and the surface of the heart, for example by means of a chemical tissue-growth facilitator (e.g., tissue growth factor (TGF)), a roughened surface of heart-interface portion 484, and/or a mechanical coupler (such as a suture or a hook that securely engages the myocardium).

Although for some applications tissue growth into the heart-contacting surface of heart-interface portion 484 is encouraged, tissue growth that reaches beyond inner wall 80, i.e., into contact with band 68, is typically discouraged. Thus, inner and outer walls 80 and 72 are typically sealed together, e.g., at their upper and lower edges.

For some applications, in addition to or instead of the functionality described hereinabove with respect to heart-interface portion 484, the heart-interface portion comprises one or more sensing electrodes (e.g., ECG electrodes), one or more electrodes suitable for cardioversion or defibrillation, and/or one or more electrodes suitable for applying pacing pulses or non-pacing pulses to the heart. Typically, but not necessarily, heart-interface portions used for these purposes are distinct from heart-interface portions used for facilitating attachment.

It is noted that, for some applications, sensing electrodes of heart-interface portion 484 serve one or both of the following purposes: (a) allow local and/or global synchronization of force application to the heart's natural rhythm, so as to optimize the ejection of blood from the heart, and (b) indicate to control unit 24 the body's overall oxygen need, as reflected by the heart rate.

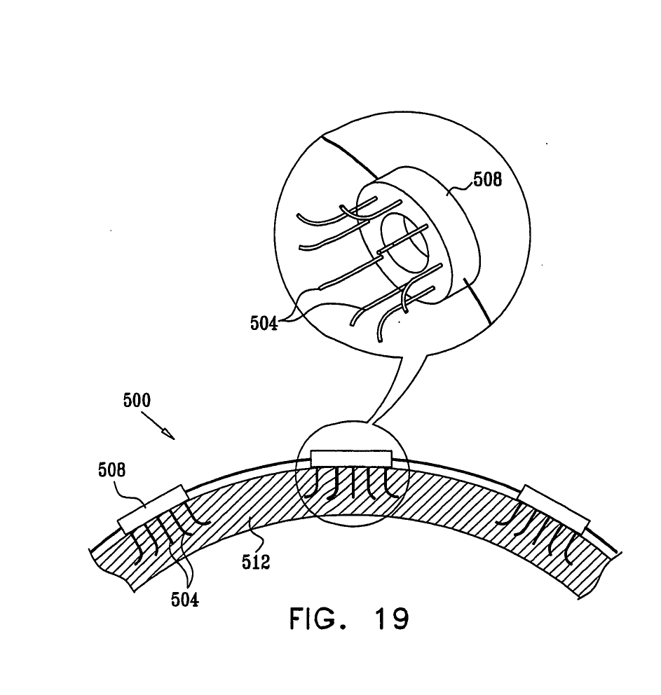

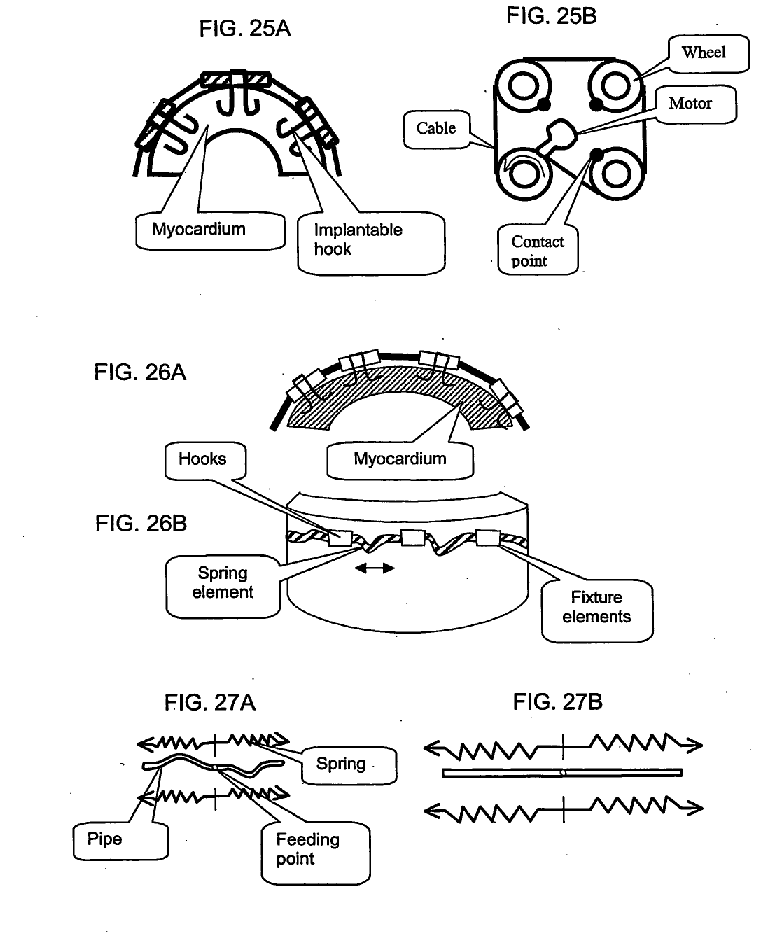

FIG. 19 is a schematic, partially cross-sectional illustration of an attachment mechanism 500 for attaching inner wall 80 to the myocardium 512 of heart 40, in accordance with an embodiment of the present invention. Attachment mechanism 500 typically comprises one or more hook assemblies, each comprising a hook assembly body 508 and one or more hooks 504. As appropriate, techniques described hereinbelow with reference to FIGS. 25A, 25B, 26A, and 26B, may be utilized in this embodiment.

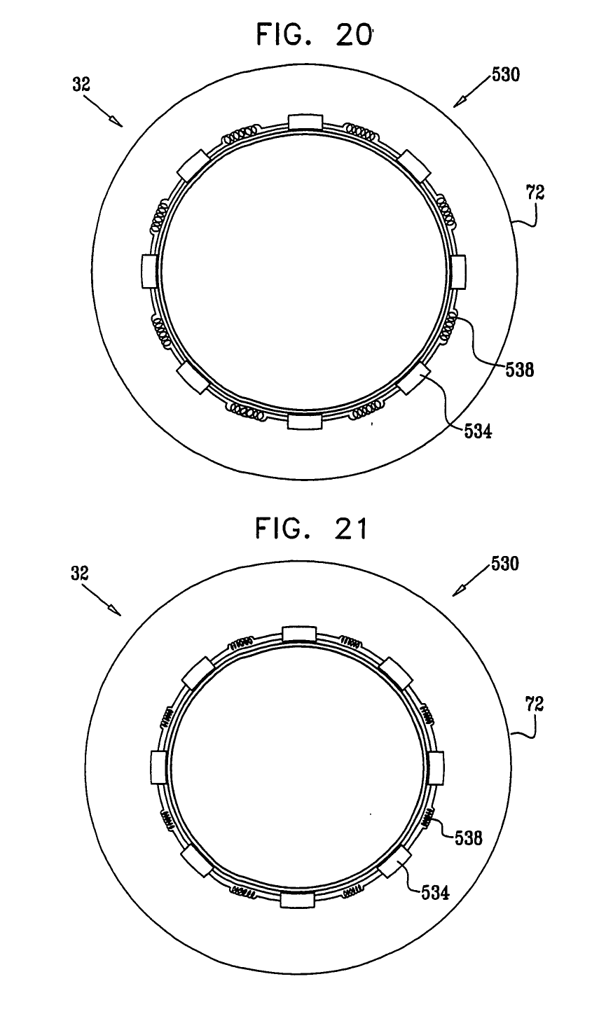

Reference is now made to FIGS. 20 and 21, which are schematic illustrations of diastolic and systolic phases, respectively, of a diastole-support mechanism 530, in accordance with an embodiment of the present invention. Active elements 534 of mechanism 530 typically comprise balloons, pistons, piezoelectric elements, or any of the other apparatus described herein for enhancing contraction of the heart, and are typically activated as described herein.

During activation of active elements 534 to assist contraction, energy is stored in one or more passive elements 538. Passive elements 538 may comprise, for example, springs, which store energy by length change, elongated elastic elements which store energy through bending, or any other passive energy storage element known in the art. For some applications, passive elements 538 are separate from active elements 534 (as shown). Alternatively or additionally, passive elements 538 are integrated with active elements 534, or inherent in the construction of active elements 534. In an embodiment, balloons, piston/cylinder arrangements, or electromechanical actuators described herein may surround, be surrounded by, or be adjacent to one or more passive elements 538.

As described, energy is stored in passive elements 538 during systole. During diastole, energy is released from the passive elements, whereby the expansion of the heart is augmented, and increased blood fills one or more of the heart's chambers. Typically, the enhanced diastolic filling of the heart is facilitated by a level of mechanical attachment between the heart and inner wall 80 that is sufficient to support the outwardly-directed force applied to the endocardial tissue, without loosening.

For some applications, passive elements 538 are replaced by active diastolic-supporting elements, which actively drive the volume of the heart to increase, to support diastolic filling.

FIG. 22 is a schematic illustration of a diastole-support mechanism 550, in accordance with an embodiment of the present invention. Diastole-support mechanism 550 is similar to diastole-support mechanism 530 described hereinabove with reference to FIGS. 20 and 21. Active elements 534 comprise balloons 64, in the embodiment of FIG. 22. Each passive element 538 comprises an elongated, slightly curved elastic element, in the embodiment of FIG. 22. Each passive element 538 is typically firmly mounted to inner wall 80 and/or between adjacent balloons 64. During systole, energy is stored in the passive elements. During diastole, the relaxation of the passive elements causes inner wall 80 (FIG. 18) to apply an outwardly-directed force to heart 40.

It is noted that diastole-support mechanisms 530 and 550 typically support the heart during cardiac systole, as well. In some modes, and/or in some patients, diastole-support mechanisms 530 and 550 are configured to support diastole, generally in the absence of a need to substantially support systolic compression of the heart.

Some embodiments of the present invention have a natural tendency to allow the heart to continue to beat almost entirely unencumbered in the event of a mechanical failure or other failure of the implanted apparatus.

For some applications, apparatus shown in the figures is applied to the heart at an angle different from that shown in the figures. Alternatively or additionally, apparatus components may be aligned within the apparatus at an angle other than that shown. For example, balloons and piston/cylinder arrangements may be applied at angles perpendicular to those shown, or at a range of angles other than those shown in the figures. Similarly, bands shown in the figures as being aligned in one direction with respect to the heart may, alternatively or additionally, be aligned in another direction.

The following is a simplified mathematical description of a hypothesized principle of action of some embodiments of the present invention:

Since the device is wrapped around the heart chamber and the balloons, any increase or decrease in the perimeter of the balloons will directly pull or release the strap wrapped around the heart chamber. The change in heart perimeter is generally equal to the summed change in perimeters of the balloons. Therefore:

2πdR=2Nπdr

Thus:

dR=Ndr

This is substantially true in every cross section of the heart operated upon by the balloons.

For simplicity, it is assumed that the heart's volume is substantially cylindrical, and thus the following equations relate to the volume relationship:

Vo—the volume ejected from the heart chamber

Vi—the total volume inserted to each of the N balloons

$$\begin{matrix}Vo ∼ L Π R {0}^{2} - L Π {R}^{2} \\ = L Π ( R {0}^{2} - R {0}^{2} + 2 R 0 dR - {dR}^{2} ) \\ = Π L ( 2 R 0 dR - d {R}^{2} ) \\ Vi = L Π {r}^{2} - L Π {r0}^{2} \\ = Π L ( r {0}^{2} + 2 r 0 dr + {dr}^{2} - r {0}^{2} ) \\ = Π L ( 2 r 0 dr + {dr}^{2} )\end{matrix}$$

It is assumed that the heart chamber length that is affected is approximately L (the base-apex length of the band), and that the volume in the affected region can be approximated by a cylindrical model.

The total volume ratio (ejected volume vs. total balloons volume) is therefore:

Vo/(NVi)=πLdR(2R0−dR)/[NπLdr(2r0+dr)]

Since dR=N dr:

Vo/(NVi)=(2R0−dR)/(2r0+dr)

Typically, the change in chamber radius is significantly smaller than the radius itself (i.e., dR<<R0).

In one example, the balloons start from a nearly collapsed state (i.e., r0˜0), and thus:

Vo/(NVi)˜2R0/dr

In another example, the balloons start at a radius which is substantially bigger than the change in their radius (i.e., dr<<r0), and thus:

Vo/(NVi)˜R0/r0

As appropriate for any given application, various number of balloons (N) and initial and final balloon volumes may be chosen in order to optimize for energy loss (friction or viscosity), total fluid volume to be transferred, etc.

It is apparent that significant volume ratios may be obtained by using balloons with small radius. For example, the radius of a diseased heart's chamber may be approximately 5 cm, while the radius of each balloon may be selected to be up to a few millimeters. It is thus clear that volume ratios may reach several times and up to tens of times.

Several theoretical examples are shown in Table III, for a model having a heart chamber radius (R0) of 5 cm, a heart chamber length (L) of 8 cm, an ejected volume (Vo) of 50 cc, and a change in heart radius (dR) of 0.203 cm. The model includes a total of N=8 balloons, each undergoing a radius change (dr) of 0.0254 cm.

TABLE III

| Balloon initial | Total inserted volume | |
|---|---|---|
| radius (r0, cm) | (N Vi, cc) | Volume ratio |
| 0 | 0.130 | 385.75 |
| 0.1 | 1.150 | 43.46 |
| 0.3 | 3.192 | 15.66 |
| 0.5 | 5.233 | 9.55 |

In some embodiments described herein, a mechanism is shown for volume amplification, which moves a small volume in and out of a shape-changing member, such as a substantially non-distensible flexible balloon or a piston/cylinder arrangement, in order to induce shortening of a heart chamber's perimeter, and thereby eject a volume of blood from the heart that is significantly larger than the volume used to fill the shape-changing member.

As opposed to a variety of compression techniques, the proposed mechanism in some of these embodiments may reach theoretically any desired ratio of the filling volume to the output volume. The mechanism converts filling volume into perimeter shortening and, in turn, the perimeter shortening produces volume ejection from the heart. For example, this may be done by placing one or more (N) balloons around the heart, each balloon may, for some applications, be located along the entire length (L) of base-apex axis.

In a sample case, a heart has a combined radius (R0) of 4.5 cm for both diseased ventricles. In order to produce a desired cardiac output, the ejected volume Vo should typically be more than 50 cc. Assuming a base-apex length of about 8 cm, the radius should change from 4.5 cm to about 4.1 cm. By using 8 balloons (as described hereinabove), each balloon should change its radius by only about 4 mm/8, i.e., by 0.5 mm. In terms of volume, if each initial balloon radius is substantially 0, then the inserted volume Vi for each balloon is less than 0.1 cc, and the total inserted volume (to 8 balloons) is less than 1 cc. This produces a volume ratio of over 50.

In another example, where lower friction is expected between each balloon and its surrounding band, each balloon's initial radius is 2 mm, and this is increased to 2.5 mm to facilitate contraction of the heart. In this case, an approximately 0.7 cc volume increase in each balloon yields a total inserted volume (over 8 balloons) of about 5.6 cc. This produces a volume ratio of over eight.

This overall concept may be actualized using various balloon configurations, including various numbers of balloons, orientations of the balloons, initial volumes of the balloons, etc., thus allowing optimization of parameters such as friction, system volume (due to the high volume ratio), and energy consumption.

Devices built in accordance with some embodiments of the present invention enable the production of desired ejection volumes while using small fluid driving volumes to cause energy transmission from an actuator to the heart chamber.

In an embodiment, energy is consumed at about 3 to about 15 W (e.g., approximately 10 W), at about 5 to about 24 V (e.g., approximately 10 V). Typical currents range between about 200 mA and about 2000 mA (e.g., approximately 1000 mA). In an embodiment, an implanted Li-Ion 1.2 AH battery is used (typically about 120 cc and 100 grams), and allows more than 1 hour of independent operation prior to recharging.

In an embodiment, during each cardiac cycle, the device moves a fluid volume of about 3 to about 15 cc (e.g., approximately 10 cc) in the pump, thus overall system size is small, typically ranging from about 80 to about 500 cc (e.g., approximately 300 cc), not including tubes and sleeve.

Materials as are known in the art are typically selected for durability, reduced friction, and biocompatibility.

The combination of small volume transport in an energy efficient system is desirable in order to enable fully implantable solutions which do not incorporate blood contact, as a fully implantable solution typically should meet one or more (or all) of the following specifications:
it should have no skin crossing elements, and thus typically should be battery operated
it should utilize a battery that is nearly continuously charged (e.g., by an externally applied electromagnetic field)
it should allow time gaps between charges that are as long as possible (e.g., to allow the user to be without an external charging unit for one or more hours)
the total volume of the implantable system (including energy source, actuators, machinery, etc.) should typically be less than 500 cc, or even 300 cc
the total mass of the implantable system (including energy source, actuators, machinery, etc.) should typically be less than 500 g, or even less than 300 g
portions of the implanted system that are in direct contact with the beating heart and move with the beating heart (typically, the sleeve and its contents) should have a mass less than about 100 g, and, for some applications, less than 50 g.

Embodiments of the present invention, such as those described herein with reference to each of the figures, typically incorporate some or all of these criteria.

Although some embodiments of the present invention are described herein as employing one or more balloons to drive the compression of sleeve 32 around heart 40, it is to be appreciated that the scope of the present invention includes the use of another mechanical actuator, in addition to or instead of a balloon. For example, such a mechanical actuator may comprise a hydraulic actuator (such as a piston/cylinder arrangement), or an electromechanical actuator (such as a piezoelectric actuator).

In the context of the present patent application and in the claims, the word “cylinder,” in the context of operation of a piston with a cylinder, is not limited to a container with a circular cross-section.

It is to be appreciated that the scope of the present invention includes replacing hydraulic actuators, which are described herein with respect to some embodiments, with non-hydraulic actuators (such as electromechanical actuators), mutatis mutandis.

It is further to be appreciated that words such as “inflating” and “expanding” as used herein are generally interchangeable, and relate to increasing the effective size of an object. Thus, for example, a piston/cylinder arrangement may be “inflated,” meaning that fluid work is used to drive the piston out of the cylinder.

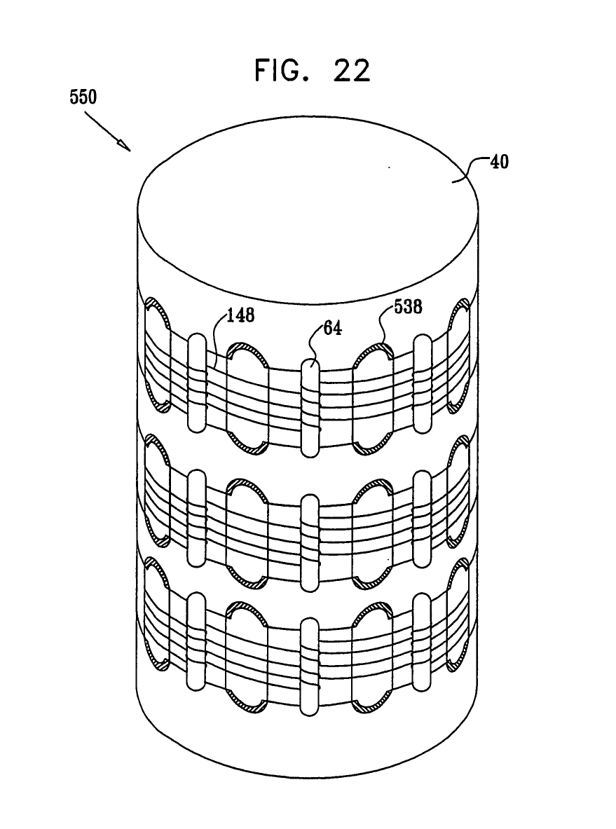

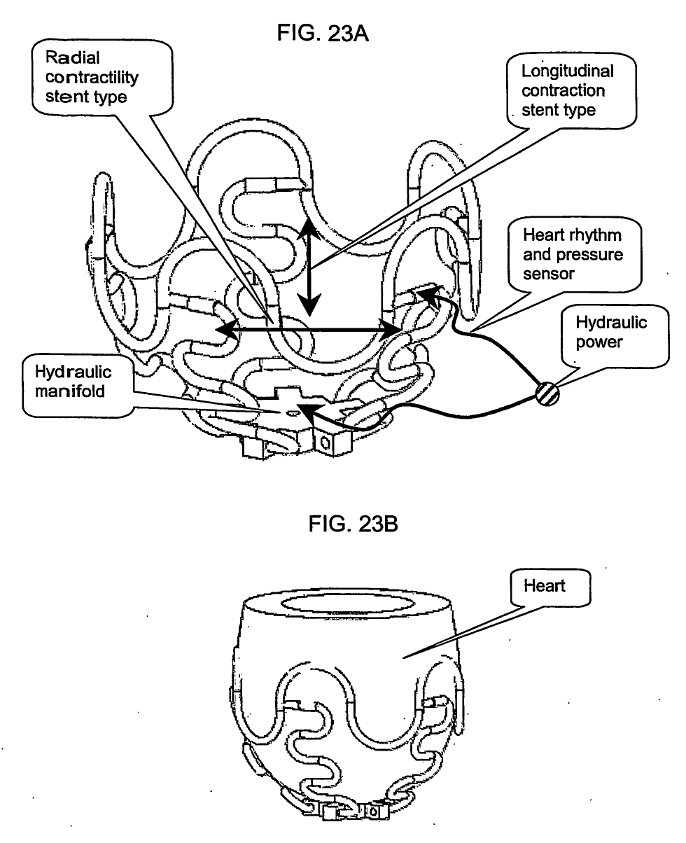

Although the following description relates primarily to FIGS. 23A to 46H, the scope of the present invention includes combining the techniques described hereinbelow with embodiments of the present invention described hereinabove with reference to FIGS. 1-22.

Reduced ventricular function and symptomatic heart failure are caused by reduced myocardial contractility and secondary neuro-hormonal peripheral vascular changes. The main cause of congestive heart failure syndrome is prior regional or global myocardial damage, which impairs the ability of the heart to contract and to sustain systemic pressure sufficient to maintain organ perfusion. Systolic heart failure syndrome thus results from impaired myocardial contractility. Patients suffering from this syndrome experience symptoms of various severities, including shortness of breath, weakness and inability to perform daily activities, and pulmonary congestion/edema and death. Cardiac medications and/or implantable assist and/or pacing devices are generally of limited efficacy in restoring systolic heart function, and generally cannot normalize left ventricular heart contractility.

In an embodiment of the present invention, a dynamic external myocardial stent (DEMS) device substantially augments myocardial contractility and restores myocardial function in heart failure patients. The DEMS device typically contracts in full synchronization with the cardiac cycle. The DEMS device is typically adapted to surround the anterior and inferior apical regions of the heart. The DEMS device is configured to dynamically contract, in synchronization with the cardiac cycle, either regionally or globally, so as to substantially augment global heart function in order to compensate for massive regional impairment of myocardial contractility.

The device restores heart function by compensating for impaired myocardial contractility because of previous massive myocardial infarction and/or any myocardial damage that caused significant impairment of global or regional systolic contractility.

Application of the DEMS device to a heart with reduced systolic function generally results in a substantial increase in cardiac contractility, an increase in the global and regional ejection fraction, and increased left ventricular systolic pressures and aortic flow. The DEMS device generally causes a substantial improvement in myocardial contractility without substantial adverse effects, because the device is implanted externally to the heart, and is therefore not in direct contact and/or interaction with any blood elements and/or with the endocardial surface of the heart.

Use of the DEMS device typically:
improves regional and global systolic performance;
restores systemic blood circulation;
improves diastolic function; and
restores left ventricular end diastolic pressure by improving systolic function and diastolic relaxation (e.g., restores “Frank-Starling” relations), and thus reduces pulmonary congestion.

The DEMS device typically comprises:
at least one preloaded element, such as a spring, a stent, a balloon, or another structure. The device typically comprises plastic, Nitinol (memory shape metal), stainless steel, a tubular mesh, or a smooth structure;
flexible tubes that are anchored into the preloaded element, and are configured to create movement of the segment of the DEMS device back and forth during diastole and systole;
an external power motor, such as a hydraulic pump, a pulse generator, or another energy device;
an apical anchoring disc that connects the branches to the hydraulic or mechanical pump or motor; and
sensing electrodes, adapted to enable synchronization of the pump to the actual heart rhythm, and contractility and pressure sensors, adapted to enable synchronization of the blood pressure in the heart with the contractibility.

Reference is made to FIG. 23A, which is a schematic illustration of a stent-type harness, and to FIG. 23B, which is a schematic illustration of the harness applied to a heart, in accordance with an embodiment of the present invention. Each tubular structure contains a balloon. The balloon is typically inflated using a hydraulic pump. The stent is in a preloaded position. When each balloon is inflated, the spring straightens, and the balloon forces the preloaded stent to straighten and enlarges the harness. Repeated application of hydraulic force creates a contraction movement, which improves the condition of the patient suffering from systolic heart failure.

### General Description of the DEMS Device

The DEMS device comprises multiple segments, corresponding to the segments of the heart on which the device is operating. The operation of each segment is fully synchronize with the heart function. Each segment is adapted to contract and relax in the range of 1-100 mm. The segments are assembled together into a harness that has a manifold that applies force to each segment fully timed and synchronized with the heart.

The force segment comprises two components. The first component is a spring-response type (quick-response) segment, such as a linear spring piston, shaped spring, etc. The spring body may comprise a material such as all types of stainless steel, Nitinol, Chrome-Nical, plastic, etc. This component should be configured at the energy-free stage; the range of force should be between 0.1 N to 100 N. The second component of the force segment provides energy to the spring force. The response time is lower, and the body material may comprise all types of materials (metal, plastic, etc.). The range of force is typically the same as the spring-response segment. The segment option is described in detail bellow.

The harness is assembled with the force segment and the structure. The force segment has quick or permanent connectors that are attached to the harness.

The manifold functions as a timing system that directs the power/energy/force to the segments with the required timing. The manifold has the appropriate number of inlets and outlets from the pump and segment.

The pump or other power source is connected to (a) the harness and (b) an implantable ECG controller and one or more pressure sensors for the left and right ventricles.

The power supply comprises a charger or rechargeable battery or other energy source, such as a biological energy source.

Together, the harness applies contraction and relaxation in a synchronized mode.

### The Anchoring Method

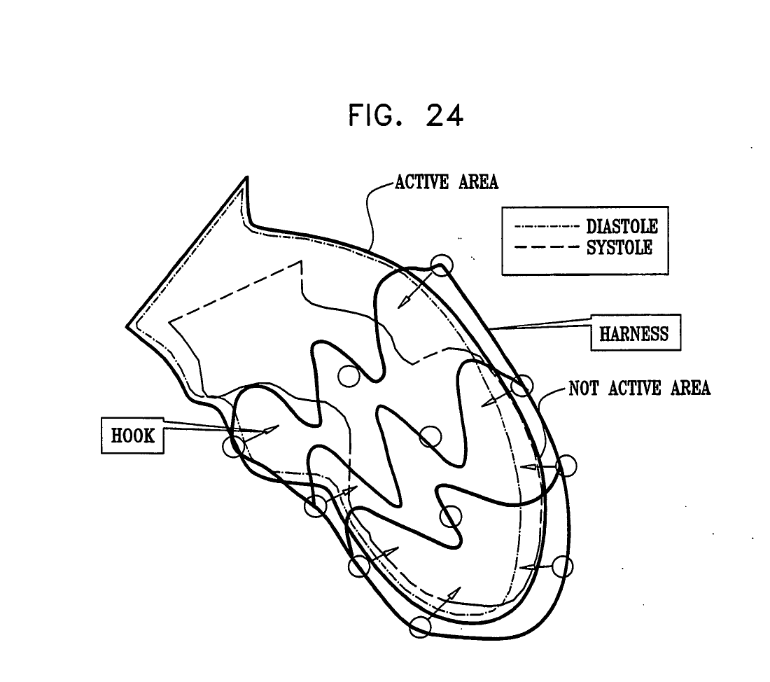

Reference is made to FIG. 24, which is a schematic illustration of an insert, in accordance with an embodiment of the present invention. The DEMS device must be held in place during operation thereof. Options include, but are not limited to, those described in this paragraph. In general, the device is anchored in the myocardium. In typical patients there are areas of the myocardium that are not functional, mainly from the apex of the heart and up. As shown in FIG. 24, one end of the insert is implanted on the myocardium at all necessary points, and the other side of the insert is anchored on the heart. Alternatively, the harness is held with a belt between the harness and the aortic arch, pulmonary artery, and/or neck.

### The Procedure

The DEMS device is adapted to be implanted using either (a) a surgical (e.g. epicardial) open-chest and/or thoracoscopic approach, or, alternatively, (b) using a percutaneous approach using either an anterograde transeptal or retrograde trans-aortic approach for endocardial implantation. For some applications, a delivery apparatus is used to deliver the myocardial stent, pre-mounted on its distal end to the target zones to facilitate proper implantation.

### Mechanical Segment Option for Systolic Heart Failure

Reference is made to FIGS. 25A and 25B, which are schematic side-view and top-view illustrations, respectively, of a cable-contraction device implanted in the myocardium, in accordance with an embodiment of the present invention. As seen in FIG. 25A, the device is attached to the myocardium with two or more hooks. The device comprises two or more implantable wheels, and a steel cable that is looped around the wheels. In the center there is a motor, e.g., a piezoelectric motor. The motor rotates the wheels, so that the cable pulls the wheels toward each other. This movement is synchronized with the heart, causing the heart to contract.

### Spring-Versus-Hydraulic Load Devices

Reference is made to FIGS. 26A and 26B, which are schematic side-view and top-view illustrations, respectively, of a spring-versus-hydraulic load device implanted in the myocardium, in accordance with an embodiment of the present invention. Heart contraction can be described as quick contraction and relatively slow relief (refill). A spring-versus-hydraulic load is the best analogy and stimulation mechanism. A first part of the spring-versus-hydraulic load device is preloaded, and a second part is an initiated load. This initiated load is typically, but not necessarily, an air load, a hydraulic load, or an electrical loads. Several options for achieving this spring-versus-hydraulic load effect are described hereinbelow, with reference to FIGS. 27A-35B.

FIGS. 27A and 27B are schematic illustrations of a pipe element in contracted and expanded positions, respectively, in accordance with an embodiment of the present invention. When hydraulic liquid is applied to the pipe, the pipe straightens and releases the force around the myocardium, as shown in FIG. 27B. Releasing the pressure causes the spring to contract to its original position, as shown in FIG. 27A. The pressure level controls the level of contraction.

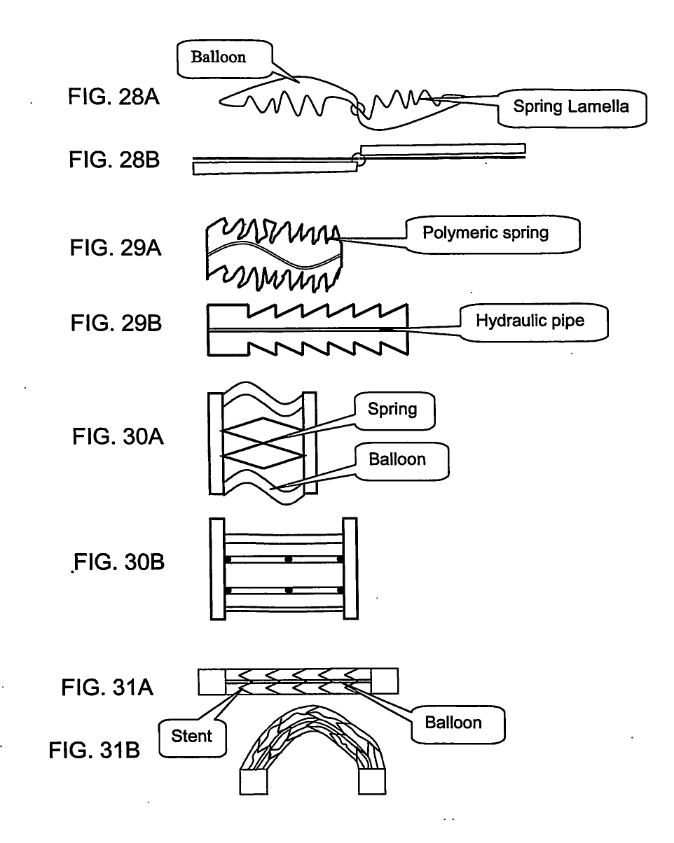

FIGS. 28A and 28B are schematic illustrations of a lamella-shaped element in contracted and expanded positions, respectively, in accordance with an embodiment of the present invention. When pressure is applied to the lamella, the lamella straightens, as shown in FIG. 28B. Reducing the pressure controls the level of contraction.

FIGS. 29A and 29B are schematic illustrations of a pipe-in-a-polymeric-spring element in contracted and expanded positions, respectively, in accordance with an embodiment of the present invention. When hydraulic force is applied to the element, the spring straightens, as shown in FIG. 29B. Releasing the pressure controls the level of contraction.

FIGS. 30A and 30B are schematic illustrations of a diamond-spring element in contracted and expanded positions, respectively, in accordance with an embodiment of the present invention. Each segment of the diamond-spring element contracts individually. The spring keeps the device in a contracted position, and the balloon straightens the device. Releasing the pressure controls the level of contraction.

FIGS. 31A and 31B are schematic illustrations of a stent-type spring element in expanded and contracted positions, respectively, in accordance with an embodiment of the present invention. The spring has a pre-shape spring load. A balloon is inside the spring. Application of pressure straightens the stent, as shown in FIG. 31A. Releasing the pressure shapes the spring.

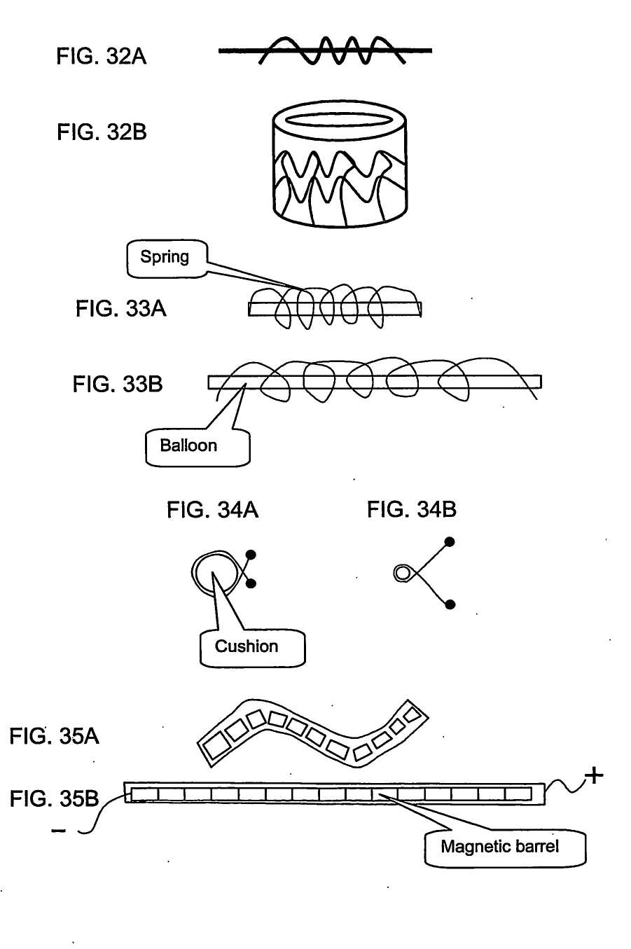

FIGS. 32A and 32B are schematic illustrations of pressure-and-vacuum elements, in accordance with embodiments of the present invention. The tubes contract or expand when a vacuum or pressure is applied, respectively.

FIGS. 33A and 33B are schematic illustrations of a helix spring element in contracted and expanded positions, respectively, in accordance with an embodiment of the present invention. The balloon pulls the helix spring element to its relief position.

FIGS. 34A and 34B are schematic illustrations of a cross spring element in contracted and expanded positions, respectively, in accordance with an embodiment of the present invention. Hydraulic or another force is applied to the cushion, thereby moving and contracting the ends of the spring.

FIGS. 35A and 35B are schematic illustrations of a polymer-magnetic element in contracted and expanded positions, respectively, in accordance with an embodiment of the present invention. The element comprises a roll of pre-shaped polymer, containing therein a small barrel of magnetic material. When current is applied through the magnet, the element straightens immediately, as shown in FIG. 35B. When the current is discontinued, the element shrinks to its original position, as shown in FIG. 35A. By changing the polarity of the magnet field it will change and will give change of shaping to activate the contraction and relief mechanism.

### Push-Pull Bars

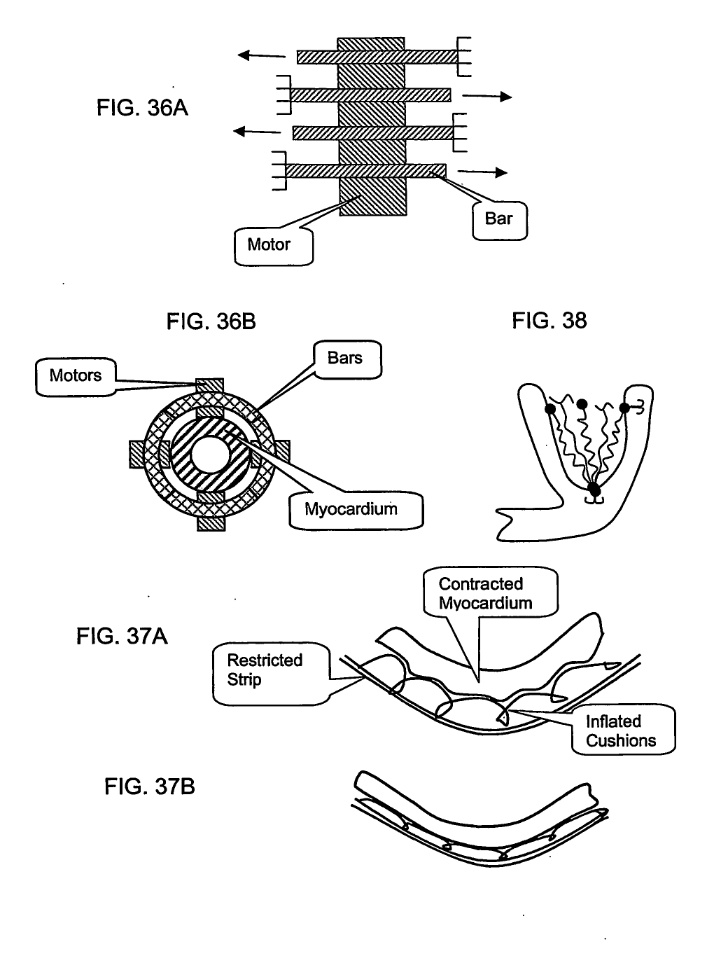

Reference is made to FIGS. 36A and 36B, which are schematic illustrations of a push-pull motor configuration, in accordance with an embodiment of the present invention. The motor holds at least two motion units. Each motion unit works in an opposite direction, such that each bar is moving in and out in the opposite direction. This movement creates the contraction and release motions. The motor is optionally a piezoelectric motor.

### Air or Liquid Cushions

Reference is made to FIGS. 37A and 37B, which are schematic illustrations of an air or liquid cushion configuration, in accordance with an embodiment of the present invention. A strip of air or liquid cushions is applied around the myocardium. A restriction strip limits the inflation direction. When the cushions are inflated, the myocardium contracts. Releasing the pressure controls the time of blood refilling.

### Percutaneous Systolic Heart Failure (SHF) and Diastolic Heart Failure (DHF) Spring Technique

Reference is made to FIG. 38, which is a schematic illustration of a percutaneous method for implanting a stent or spring, in accordance with an embodiment of the present invention. The stent or spring is adapted to prevent the rupture of the myocardium wall by reducing the load on the wall to help treat SHF problems. The device is typically delivered for implantation on a catheter delivery system. The mechanism hooks the device to the myocardium and puts the device in place. This device helps the myocardium wall to enlarge, but does not help with systolic action. This method is noninvasive.

### Inflating Harness

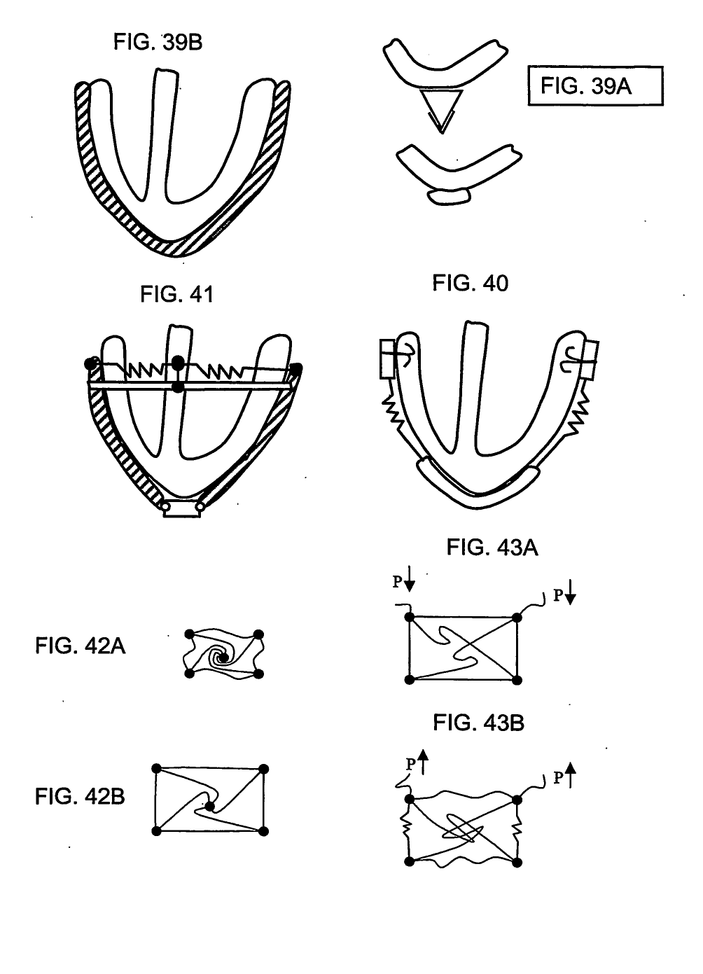

Reference is made to FIGS. 39A and 39B, which are schematic illustrations of an inflating harness, in accordance with an embodiment of the present invention. The heart is covered with a harness net. The net is fit to diastolic stage (at relaxation) before systolic stage (contraction). The cushions are connected to the net, and are adapted to contract the heart at the contraction phase. The inflated cushion is typically configured to assume a triangle shape when pressure is applied thereto, in order to create the contraction motion. When pressure is not applied, the cushion relaxes and assumes an elliptic shape having a minimum size. This cushion covers the damaged area of the heart, such that the contraction motion helps to pump blood to the body.

### Apex to Aorta Contraction Direction

Reference is made to FIG. 40, which is a schematic illustration of an inflating spring, in accordance with an embodiment of the present invention. The inflating spring pulls the bottom cover up and causes contraction motion in the axial direction; simultaneously, radial contraction occurs.

### Contour of a Specific Heart

Reference is made to FIG. 41, which is a schematic illustration of a semisolid structure having a contour of a specific heart, in accordance with an embodiment of the present invention. Each side of the heart has a negative image of the heart. These elements are pushed against the myocardium. A spring and a balloon cause the movement.

### Spring and Balloon Back Node

Reference is made to FIGS. 42A and 42B, which are schematic illustrations of spring-and-balloon back node structure, in accordance with an embodiment of the present invention. A constant spring comprises four lamellas, which expand together. The lamellas work independently of each other, allowing a different force to be applied in each direction. A high pressure is applied to the surrounding tubes, which produce an opposing force, resulting in the contraction and relaxation of the structure.

### Center-Pressured Balloon Contraction Nodes

Reference is made to FIGS. 43A and 43B, which are schematic illustrations of a center-pressured balloon contraction node structure, in accordance with an embodiment of the present invention. The structure comprises four nodes out of many other on the net (representing a complete device). The frame comprises strong wire. The structure further comprises a crossed element, which comprises tubes containing liquid. Pressure applied in the tube causes the crossing tube to reshape, resulting in the contraction motion. Releasing the pressure releases the force applied to the heart.

### Circumference-Pressured Balloon Contraction Nodes

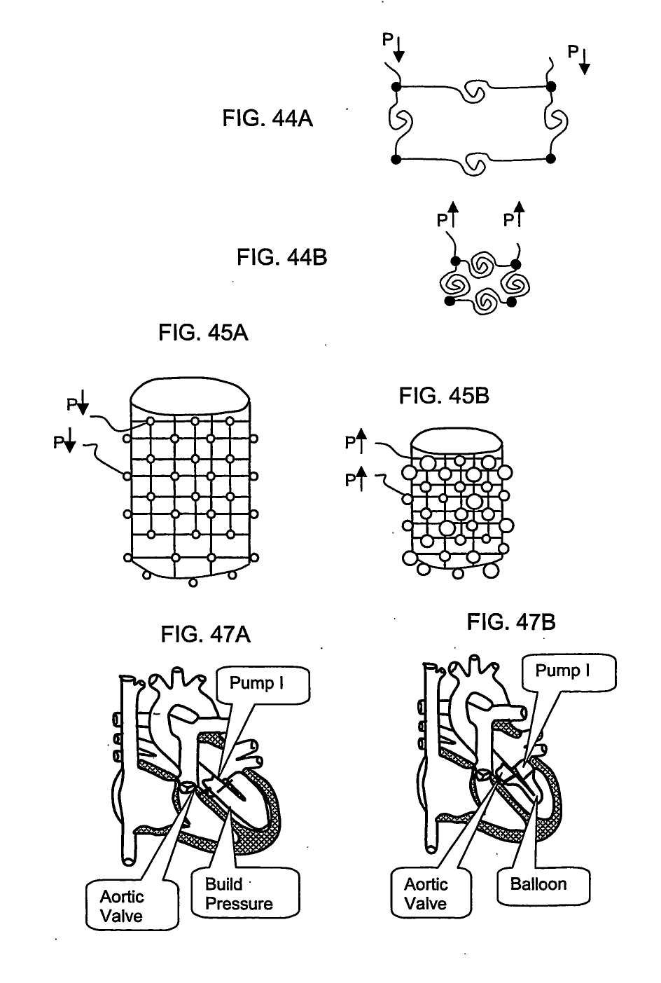

Reference is made to FIGS. 44A and 44B, which are schematic illustrations of a circumference-pressured balloon contraction node structure, in accordance with an embodiment of the present invention. This embodiment is similar to the center-pressured balloon contraction node structure described hereinabove with reference to FIGS. 43A and 43B, except that the tubes contract around the nodes, and not across the center of the structure.

### Balloon in a Loop Contractions

Reference is made to FIGS. 45A and 45B, which are schematic illustrations of a balloon-in-a-loop contraction node structure, in accordance with an embodiment of the present invention. A harness covers the heart. The harness is divided into widthwise and lengthwise strips, which together divide the harness into sections. A loop is located in the center of each section. A balloon is placed in each loop, such that when the balloon is inflated, the circumference of the balloon increases, thereby shortening the strip. Inflation and deflation of the balloons reduce the harness size when the heart contracts and relaxes according to its rhythm. This system is synchronized with heart activity.

### Non-Invasive Assist Device

Reference is made to FIGS. 46A-H, which are schematic illustrations of a non-invasive assist device, in accordance with an embodiment of the present invention. The device comprises four main components: (a) the delivery system, (b) the device, (c) the controller and loading, and (d) the power supply. The delivery system is similar to an implantable ICD.

For some applications, a transvenous approach is used. The physician makes a small incision near the collarbone and maneuvers one or more leads through a vein into the heart. The tip of each lead (the electrode) is positioned next to the endocardium. The device is then implanted under the skin in a specially prepared pocket, usually in the right or left upper chest. Alternatively, sternotomy is used. This approach is similar to a thoracotomy, however, the incision is made over the breastbone, or sternum, and the leads are advanced into the heart. This is the type of operation that is commonly used in coronary bypass and heart valve surgery.

All four balloon bases may be used with all configurations.

### Non-Invasive Electrical and Pump Assist Device

Reference is made to FIGS. 47A and 47B, which are schematic illustrations of a non-invasive electrical and pump assist device, in accordance with an embodiment of the present invention. The electrical pump base assist device comprises a balloon valve and a pump. This device works simultaneously with other functional portions of the heart. This device is implanted using a procedure similar to that typically used for ICD implantation.

In an embodiment, the device comprises a pump adapted to replace the mitral valve and the aortic valve, and a balloon placed between them. The pump pumps blood to the balloon, so that the balloon enlarges and builds pressure inside, until the pressure in the balloon exceeds the aortic pressure. The balloon then pushes the blood to the aorta until the pressures equalize.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for compressing at least one chamber of a heart of a patient's body, the apparatus comprising:
one or more inflatable elements;
a pump in fluid communication with the inflatable elements; and
at least one band having a first one or more first portions and second one or more second portions, the first and second portions alternatingly arranged,
the first one or more first portions and second one or more second portions having respective variable first and second total lengths,
the first one or more first portions adapted to be placed around at least a portion of the heart in mechanical communication with the portion of the heart, and
each of the second portions placed around at least 180 degrees of a periphery of at least one of the inflatable elements, such that the second portions are in mechanical communication with the heart via the first portions, and such that when the inflatable elements are inflated by the pump the first total length decreases by an amount that the second total length increases.

2. The apparatus according to claim 1, wherein the first one or more first portions are adapted to be disposed between the heart and the one or more inflatable elements.

3-4. (canceled)

5. The apparatus according to claim 1, wherein the pump is adapted to pump a liquid to inflate the inflatable elements.

6. The apparatus according to claim 1, wherein the pump is adapted to pump a gas to inflate the inflatable elements.

7. The apparatus according to claim 1, wherein, for each inflatable element, only one second portion is placed around at least 180 degrees of its periphery.

8. The apparatus according to claim 1, wherein the inflatable elements are coupled to the band such that when the first one or more first portions are placed around the portion of the heart, the inflatable elements are symmetrically disposed around the heart.

9. The apparatus according to claim 1, wherein the inflatable elements are coupled to the band such that when the first one or more first portions are placed around the portion of the heart, the inflatable elements are asymmetrically disposed around the heart.

10-13. (canceled)

14. The apparatus according to claim 1, comprising an inner layer, adapted to be disposed between the band and the heart, and at least one hook, adapted to secure the inner layer to the heart.

15. The apparatus according to claim 1, comprising a diastole-supporting mechanism, adapted to store energy from the pump during systole, and to release the energy during diastole in a manner that facilitates application of an outwardly-directed force to an epicardial surface of the heart during diastole.

16. The apparatus according to claim 1, wherein the at least one band comprises a plurality of bands.

17-20. (canceled)

21. The apparatus according to claim 1, wherein the apparatus comprises an apical-region cover, coupled to the band and adapted to cover a region in a vicinity of an apex of the heart.

22. The apparatus according to claim 21, wherein the apical-region cover is adapted to be disposed on the heart such that the vicinity of the apex of the heart does not include the apex.

23. (canceled)

24. The apparatus according to claim 21, wherein the apical-region cover is adapted to passively apply a compressive force to the vicinity of the apex of the heart.

25. (canceled)

26. The apparatus according to claim 21, wherein the apical-region cover is adapted to actively apply a compressive force to the vicinity of the apex of the heart.

27. The apparatus according to claim 1, wherein at least one of the second portions comprises at least one flexible line, which is wrapped at least twice around the periphery of at least one of the inflatable elements.

28. The apparatus according to claim 27, wherein the at least one flexible line comprises a plurality of flexible lines, each wrapped at least twice around the periphery of the at least one of the inflatable elements.

29. The apparatus according to claim 1, wherein at least one of the second portions comprises one or more flexible lines, each flexible line adapted to be placed around at least 180 degrees of the periphery of at least one of the inflatable elements.

30-31. (canceled)

32. The apparatus according to claim 29, wherein the one or more flexible lines comprises at least 2 lines.

33-34. (canceled)

35. The apparatus according to claim 1, wherein, for at least one of the inflatable elements, at least two or more second portions are placed around at least 180 degrees of its periphery.

36. (canceled)

37. The apparatus according to claim 1, wherein the apparatus comprises a sleeve adapted for placement around the heart, and wherein the band and the inflatable elements are disposed within the sleeve.

38. The apparatus according to claim 37, wherein the band is isolated by the sleeve from contact with tissue of the patient's body.

39-40. (canceled)

41. The apparatus according to claim 83, wherein a total mass of the apparatus is less than 300 g, and wherein the apparatus comprises a battery adapted to drive the pump for at least one hour without being recharged from a source outside of the patient's body.

42. The apparatus according to claim 41, wherein the battery has a capacity of less than 2 Amp-Hour.

43. (canceled)

44. The apparatus according to claim 83, wherein a total volume of the apparatus is less than 300 cc, and wherein the apparatus comprises a battery adapted to drive the pump for at least one hour without being recharged from a source outside of the patient's body.

45. The apparatus according to claim 1, wherein each of the inflatable elements is adapted to increase in volume by at least 0.1 cc in response to the inflation by the pump.

46. The apparatus according to claim 45, wherein each of the inflatable elements is adapted to increase in volume by at least 10 cc in response to the inflation by the pump.

47. The apparatus according to claim 1, wherein each of the inflatable elements is adapted to increase in volume by less than 80 cc in response to the inflation by the pump.

48-49. (canceled)

50. The apparatus according to claim 49, wherein the exactly one inflatable element is adapted to increase in volume by at least 5 cc in response to the inflation by the pump.

51. The apparatus according to claim 1, wherein the one or more inflatable elements comprises a plurality of inflatable elements.

52-59. (canceled)

60. The apparatus according to claim 59, wherein a total increase in volume of all of the inflatable elements in response to being inflated by the pump is 25 cc.

61. The apparatus according to claim 1, wherein the apparatus is configured such that the decrease of the first total length is at least 8 mm.

62. The apparatus according to claim 61, wherein the apparatus is configured such that the decrease of the first total length is at least 40 mm.

63-64. (canceled)

65. The apparatus according to claim 84, wherein when the inflatable elements are inflated by the pump during the cardiac cycle, the peak reduction in volume of the heart is at least 1000% of the total volume of fluid pumped into all of the inflatable elements by the pump during the cardiac cycle.

66. Apparatus for compressing at least one chamber of a heart of a patient's body, the apparatus comprising:
one or more shape-changing members;
a control unit, coupled to the shape-changing members; and
at least one band having a first one or more first portions and second one or more second portions, the first and second portions alternatingly arranged,
the first one or more first portions and second one or more second portions having respective variable first and second total lengths,
the first one or more first portions adapted to be placed around at least a portion of the heart in mechanical communication with the portion of the heart, and
each of the second portions placed around at least 180 degrees of a periphery of at least one of the shape-changing members, such that the second portions are in mechanical communication with the heart via the first portions, and such that when the shape-changing members are driven by the control unit to change shape, the first total length decreases by an amount that the second total length increases.

67-68. (canceled)

69. The apparatus according to claim 68, wherein the electromechanical actuator comprises an electromagnet.

70. The apparatus according to claim 68, wherein the electromechanical actuator comprises a piezoelectric element.

71. Apparatus for compressing at least one chamber of a heart of a patient's body, the apparatus comprising:
one or more shape-changing members;
a control unit, adapted to drive the shape-changing members to change shape; and
a band, an effective length of the band being adapted to surround a portion of the heart and to shorten responsive to the control unit driving the shape-changing members to change shape, whereby to enhance contraction of the heart.

72. The apparatus according to claim 71, wherein the band is adapted to be looped around at least one of the shape-changing members.

73. The apparatus according to claim 71, wherein the band is adapted to be looped a plurality of times around at least one of the shape-changing members.

74-76. (canceled)

77. The apparatus according to claim 71, wherein at least one of the shape-changing members comprises a hydraulic actuator.

78. The apparatus according to claim 77, wherein the hydraulic actuator comprises a balloon.

79. The apparatus according to claim 77, wherein the hydraulic actuator comprises a piston and a cylinder.

80. The apparatus according to claim 79, wherein the control unit is adapted to drive fluid into the cylinder to cause the effective length of the band to shorten.

81. The apparatus according to claim 79, wherein the control unit is adapted to draw fluid out of the cylinder to cause the effective length of the band to shorten.

82. The apparatus according to claim 71, wherein at least one of the shape-changing members comprises an electromechanical actuator.

83. Apparatus for compressing at least one chamber of a heart of a patient's body, the apparatus comprising:
one or more inflatable elements;
a pump in fluid communication with the inflatable elements; and
at least one band in mechanical communication with the inflatable elements, a portion of the band adapted to be placed around at least a portion of the heart in mechanical communication with the portion of the heart,
the inflatable elements arranged such that when the inflatable elements are inflated by the pump, the inflatable elements apply more force to the heart via shortening of the portion of the band than via expansion of the inflatable elements against the heart.

84. The apparatus according to claim 83, wherein the inflatable elements are arranged such that when the inflatable elements are inflated by the pump during a cardiac cycle, a peak reduction in volume of the heart is at least 200% of a total volume of fluid pumped into all of the inflatable elements by the pump during the cardiac cycle.

85-86. (canceled)

87. The apparatus according to claim 37,
wherein a combined mass of the sleeve, the inflatable elements, and the band, including any fluid therein, does not exceed 100 g at any phase of a contraction cycle of the heart.

88. The apparatus according to claim 87, wherein the mass does not exceed 70 g at any phase of the heart contraction cycle.

89. The apparatus according to claim 88, wherein the mass does not exceed 50 g at any phase of the heart contraction cycle.
